(19) United States
(12) Patent Application Publication
(10) Pub. No.: US 2025/0346551 A1
(43) Pub. Date: Nov. 13, 2025

(54) PREPARATION AND PURIFICATION OF CIS-2-ALKENOIC ACIDS

(71) Applicant: BROMINE COMPOUNDS LTD., Beer-Sheva (IL)

(72) Inventors: Jakob OREN, Nesher (IL); Nasif YASSIN, Tamra (IL); Nicka CHINKOV, Kibbutz Mishmar Haemek (IL); Ronit YAHALOMI SEGUI, Kiryat Bialik (IL); Ari AYALON, Haifa (IL)

(73) Assignee: BROMINE COMPOUNDS LTD., Beer-Sheva (IL)

(21) Appl. No.: 18/872,348

(22) PCT Filed: Jun. 8, 2023
(86) PCT No.: PCT/IL2023/050593

§ 371 Date: Dec. 6, 2024

Related U.S. Application Data
(60) Provisional application No. 63/350,045, filed on Jun. 8, 2022.

(51) Int. Cl. C07C 51/44; C07C 45/63; C07C 45/80; C07C 51/347; C07C 51/41; C07C 51/64

(52) U.S. Cl. CPC C07C 51/44; C07C 45/63; C07C 45/80; C07C 51/347; C07C 51/41; C07C 51/64

(57) ABSTRACT

A method of purification of crude cis-2-alkenoic acid by thermal separation, comprising wiped-film evaporation or vacuum distillation of the crude cis-2-alkenoic acid, under a temperature-reduced pressure profile whereby isomerization of cis-2-alkenoic acid into trans-2-alkenoic acid is minimized.

The invention relates to the synthesis and purification of long chain cis-α,β-unsaturated acids of the formula R—CH═CH—COOH, i.e., cis-2-alkenoic acids, where R indicates an alkyl residue (linear or branched) consisting of not less than, e.g., 4 carbon atoms.

It has been reported that long chain cis-2-alkenoic acids act as bio-dispersants. For example, it was shown in WO 2008/143889 and the Journal of Bacteriology 191:1393-1403 (2009) that cis-2-decenoic acid, produced by the bacterium Pseudomonas aeruginosa, is capable of inducing P. aeruginosa and other gram-negative and gram-positive bacteria and fungi to undergo a physiologically-mediated dispersion response, resulting in the dis-aggregation of surface-associated microbial populations and communities known as biofilms.

In co-assigned WO 2020/240559, it was demonstrated that cis-2-decenoic acid can act as an effective adjunctive to bromine-containing biocides in the treatment of biofilm and planktonic bacteria in water systems and on surfaces in contact with the water, to achieve significant enhancement in the killing of bacteria in both pure and mixed cultures typically found in industrial and natural waters, relative to treatment with the brominated biocides alone. Notably, it was shown in WO 2020/240559 that satisfactory enhancement of bromine-based water treatments can be achieved with the aid of cis-2-decenoic acid of moderate purity, say, 80-95% (by gas chromatography, GC area %).

WO 2020/240559 (Preparation 3) shows purification of cis-2-decenoic acid by liquid-solid column chromatography. Experimental results reported below indicate that very high purity levels are attained by liquid-solid column chromatography, but the yield may often not be satisfactory.

For example, with silica gel as adsorbent, using saturated hydrocarbon (heptane or hexane) and an ester (ethyl acetate) as solvents, akin to Preparation 3 of WO 2020/240559, purity levels as high as 99% HPLC % area were reached, but the yield of the final product was quite low (˜25%-40% yield).

Cahiez et al., “Stereospecific syntheses of alkenyllithium reagents from alkenyl iodides”, Synthesis, 1976, 4, 245-8 report the synthesis of cis-2-decenoic acid and its purification by distillation, indicating boiling point of 102-103°/0.5 torr.

We have found that purification of cis-2-alkenoic acids such as cis-2-decenoic acid by thermal separation methods can lead to isomerization of the cis isomer into the trans isomer. If thermal separation by evaporation/distillation is not managed properly, the amount of the trans isomer can exceed 1.0% (HPLC area). However, despite the thermal lability of cis-2-alkenoic acids, process variables (e.g., reduced pressure, temperature and time) can be adjusted to suppress formation of undesired impurities driven by high temperatures. Experimental results shown below indicate that high assay (≥90%) and good purification yield (70-90%) can be achieved, especially with wiped film evaporation.

Thus, the invention is primarily directed to a method of purification of crude cis-2-alkenoic acid by thermal separation, comprising wiped-film evaporation or vacuum distillation of the crude cis-2-alkenoic acid, under a temperature-reduced pressure profile whereby isomerization of cis-2-alkenoic acid into trans-2-alkenoic acid is minimized.

The cis-2-alkenoic acid is preferably cis-2-decenoic acid and is purified by wiped-film evaporation carried out at a temperature of not less than 150° C. (e.g., 150 to 180° C.) and reduced pressure of <5 mbar (e.g., 1-5 mbar, or 2-5 mbar, e.g., 2-4 mbar) whereby isomerization of the cis isomer to the trans isomer is suppressed such that the level of the trans isomer is less than 1.0% by HPLC area. The wiped-film evaporation may include a step in which a condensate is collected and returned to the feed stream (to reach the targeted assay).

Crude cis-2-alkenoic acid obtained by various synthetic pathways can be purified by the thermal separation method of the invention (e.g., wiped-film evaporation). However, crude cis-2-alkenoic acid, prepared by a two-step process comprising bromination of 2-alkanone to give 1,3-dibromo-2-alkanone; and rearrangement of the 1,3-dibromo-2-alkanone to the cis-2-alkenoic acid, could especially benefit from purification by wiped-film evaporation. This synthetic pathway is now described in detail, in reference to our earlier patent application PCT/IL2021/051423 (≡WO 2022/118309).

The synthesis is based on a two-step process consisting of brominating the corresponding 2-alkanone to give crude 1,3-dibromo-2-alkanone as a main product alongside other isomers, followed by rearrangement of the 1,3-dibromo-2-alkanone to the unsaturated acid, depicted by the scheme below:

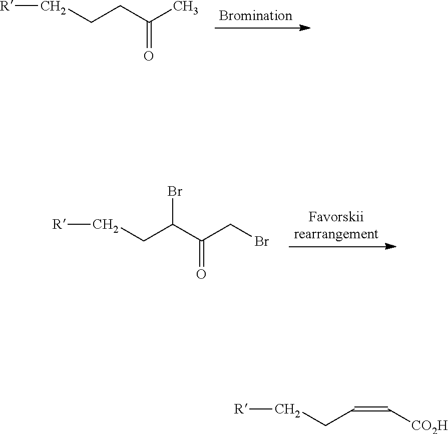

[where R′ is alkyl, e.g., C₂H₅, C₃H₇, C₄H₉, C₅H₁₁ and C₆H₁₃]. The abovementioned two-step synthesis was first described by Rappe et al. [Acta Chemica Scandinavica (1965), Vol. 19 p. 383-389]. The rearrangement took place in an alkaline environment, using alkali carbonates or alkali bicarbonates as a base. A similar approach was reported by the same research group in Organic Syntheses (1973), Vol. 53, p. 123-127.

An attempt to modify the two-step synthetic pathway is found in U.S. Pat. No. 8,748,486, where it was explained that alkali bicarbonate can only effectively advance the preparation of short chain cis-α,β-unsaturated acids. The authors reported that the rearrangement reaction of a long chain brominated ketone, e.g., 1,3-dibromo-2-decanone, was very slow in the presence of an alkali bicarbonate, and the desired fatty acid was not obtained even after prolonged reaction time. The authors switched to an alkali hydroxide to advance the preparation of long chain cis-α,β-unsaturated acids (the terms “cis-α,β-unsaturated acids” and “cis-2-alkenoic acids” are used interchangeably).

The Experimental results reported below in reference to the synthetic pathway are in line with the observations made in U.S. Pat. No. 8,748,486: rearrangement reactions of long chain 1,3-dibromo-2-alkanones under an alkaline pH barely make any progress, even at high reaction temperatures, and are prone to the occurrence of thermal runaway (a sudden and rapid rise in the reaction temperature). Such a reaction profile is unacceptable for a process running on an industrial scale.

However, it has been found that a given amount of the alkali salt of the target cis-2-alkenoic acid should be provided in the reaction mixture to advance the rearrangement reaction of the 1,3-dibromo-2-alkanone in an effective and manageable manner, leading to the cis-2-alkenoic acid. The added alkali salt of the cis-2-alkenoic acid can be supplied to the rearrangement reaction from a previous run, as shown below. With the aid of a small amount of cis-2-alkenoic acid or its salt, added at the beginning of the rearrangement reaction, a manageable process is provided.

Accordingly, the synthesis is based on a process for the preparation of a cis-2-alkenoic acid [R—CH═CH—COOH] or an alkali metal salt thereof [R—CH═CH—COOM, wherein M is an alkali metal], comprising rearranging 1,3-dibromo-2-alkanone [R—CHBr—C(O)—CH₂Br] in an alkaline environment (e.g., generated by an alkali carbonate, or alkali carbonate/bicarbonate mixture) in the presence of a catalytically effective amount of an alkali metal salt of the cis-2-alkenoic acid, and isolating from the reaction mixture the cis-2-alkenoic acid, either in the form of the free acid or in the form of the alkali metal salt (e.g., by separating the reaction mixture into aqueous and organic phases, and working-up the aqueous phase, to recover therefrom the cis-2-alkenoic acid, either in the form of the free acid or in the form of the alkali metal salt).

R is an alkyl group consisting of not less than four carbon atoms, e.g., not less than five carbon atoms, for example, R is C4-C11 alkyl. For example, cis-2-decenoic acid (R is C₇H₁₅), in the free acid form, is collected as an oil. On reaction of the so-formed cis-2-decenoic acid with an alkali hydroxide, e.g., KOH, the corresponding potassium salt is obtained as a paste-like solid.

The cis-2-alkenoic acids R—CH═CH—COOH prepared by the synthetic pathway described above, and purified by the present invention, are preferably linear. That is, R is usually a straight alkyl chain CH₃—(CH₂)ₙ— (3≤n, e.g., 3≤n≤10, 3≤n≤6). For example, the preparation of a cis-2-alkenoic acid by the rearrangement reaction of the corresponding 1,3-dibromo-2-alkanone depicted below was studied (but it should be noted that R is not limited to a normal chain, and may be a branched alkyl group, say, iso-alkyl):

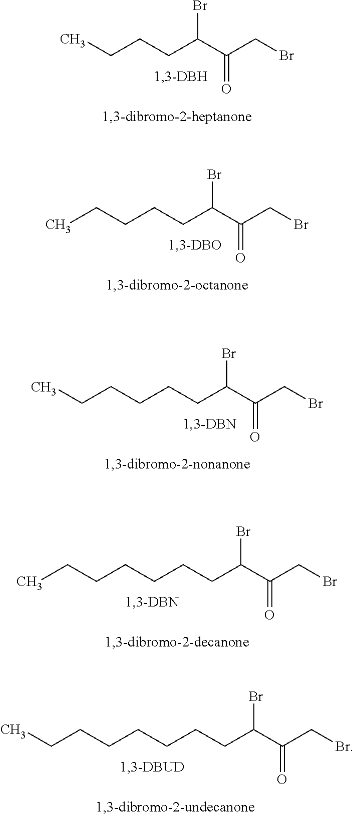

and all were found to benefit from the addition of a catalytically effective amount of the target cis-2-alkenoic acid or its alkali metal salt to the alkaline reaction mixture. By “catalytically effective amount”, it is meant that the added amount is up to 15 mol %, e.g., up 10 mol %, e.g., from 1 to 5 mol % based on 1,3-dibromo-2-alkanone.

The 1,3-dibromo-2-alkanone undergoing the rearrangement reaction is most conveniently prepared by brominating the corresponding 2-alkanone [R—CH₂—C(O)—CH₃](e.g., 2-heptanone, 2-octanone, 2-nonanone, 2-decanone or 2-undecanone) in concentrated hydrobromic acid (e.g., from 30% to 48% by weight HBr solution), by the slow addition of elemental bromine (stoichiometry dictates a ˜2:1 molar ratio of Br₂:2-alkanone).

The weight ratio of the 2-alkanone starting material to the aqueous HBr is of 1:1 to 1:2. The reaction medium is chilled to a temperature in the range from 5 to 20° C. e.g., around 5 to 10° C. Under these conditions, elemental bromine adds smoothly to the 2-alkanone, with most of the reaction occurring during the addition of the bromine; no bromine accumulation (marked by a characteristic yellow color acquired by the reaction mixture) is observed.

The bromine addition time, on a laboratory scale, is usually from 1 to 5 hours. After the addition of the elemental bromine has been completed, the reaction mixture is held at room temperature (15-25° C.), optionally under stirring, for a period of time (“hold time”). Hold time may last between 6 and 24 hours, e.g., 6 and 12 hours. Long hold times appear to be beneficial because the bromination reaction of 2-alkanone leads to a few isomeric by-products, chiefly 3,3-dibromo-2-alkanone. GC analysis of the reaction mixture indicates that the desired isomer, 1,3-dibromo-2-alkanone progressively becomes the predominant product with the passage of time, i.e., an extended hold time enables a significant interconversion of 3,3-dibromo-ketone to 1,3-dibromo-ketone.

To illustrate the importance of prolonged hold times in shifting the distribution of the isomeric mixture consisting of 1,3-dibromo-2-alkanone and 3,3-dibromo-2-alkanone in favor of the former at the expense of the latter, experimental data is tabulated in Table A, based on the procedures of brominating 2-nonanone, 2-decanone or 2-undecanone (reported in the Working Examples below):

TABLE A

| | Composition**, by GC, area % | | | | | |
|---|---|---|---|---|---|---|
| | Brominating | | Brominating | | Brominating | |
| | 2-nonanone | | 2-decanone | | 2-undecanone | |
| Hold | in HBr | | in HBr | | in HBr | |
| Time* | 1,3- | 3,3- | 1,3- | 3,3- | 1,3- | 3,3- |
| h | DBN | DBN | DBD | DBD | DBUD | DBUD |
| 0.1-0.5 | 42.8 | 27.5 | 47.5 | 26.6 | 48.1 | 26.0 |
| 2.0-2.5 | 60.3 | 21.4 | 59.9 | 13.6 | 55.9 | 17.9 |
| 20-21 | 70.6 | 6.3 | 69.6 | 5.8 | 69.4 | 6.2 |

*Time elapsed after completion of the bromine addition at T_{R} ~20° C.
**Other impurities consisting of 3-bromo-2-alkanone and isomers of tribromo-2-alkanone are also present

It is seen that the product mixtures obtained by brominating various 2-alkanones in hydrobromic acid behave in a similar manner on standing over long hold times. Initially, the mixture consisting of 1,3-dibromo-2-alkanone and 3,3-dibromo-2-alkanone is proportioned ˜2:1; after ˜twenty hours, the proportion is higher than 10:1, with the equilibrium stabilizing and reaching ˜70% (GC, area %) of the desired isomer which is amenable to the rearrangement reaction, i.e., the 1,3-dibromo-2-alkanone.

Evolution of hydrogen bromide occurs during the bromine addition and subsequent hold phases; the gas is absorbed in a suitable aqueous medium, to be collected as aqueous hydrobromic acid.

To recover the crude 1,3-dibromo-2-alkanone, the reaction mixture is worked-up by the addition of water, followed by separation into an aqueous phase (consisting of ˜48% w/w hydrobromic acid) and an organic phase, consisting of the crude product. Typically, as indicated by the data tabulated in Table A, the crude product recovered contains ˜70% (GC, area) of the 1,3-dibromo-2-alkanone.

Accordingly, the 1,3-dibromo-2-alkanone used in the rearrangement reaction is a crude 1,3-dibromo-2-alkanone obtained by the steps of:
brominating the corresponding 2-alkanone in concentrated hydrobromic acid by the addition of elemental bromine, whereby 1,3-dibromo-2-alkanone is formed in the reaction mixture alongside 3,3-dibromo-2-alkanone;
maintaining the reaction mixture over a hold time adjusted to maximize the interconversion of 3,3-dibromo-2-alkanone to 1,3-dibromo-2-alkanone (e.g., to reach >65%, >67%, >69% (GC, area %) of 1,3-dibromo-2-alkanone); and collecting the crude 1,3-dibromo-2-alkanone.

The crude 1,3-dibromo-2-alkanone, without further purification, can now proceed to the rearrangement reaction. However, the invention is not limited to the rearrangement of 1,3-dibromo-2-alkanone obtained by brominating a 2-alkanone in concentrated hydrobromic acid; other methods for the preparation of 1,3-dibromo-2-alkanones reported in the literature may be used, e.g., brominating a 2-alkanone in an organic solvent such as halogenated hydrocarbon (CH₂Cl₂ or CH₂Br₂) with the aid of acceptable bromination reagents.

A convenient way to carry out the rearrangement reaction comprises gradually adding the 1,3-dibromo-2-alkanone to a reaction vessel which was previously charged with an alkaline aqueous solution (e.g., consisting of 10 to 30% w/w Na₂CO₃, K₂CO₃ or a mixture thereof dissolved in water, or carbonate/bicarbonate mixtures) and a catalytically effective amount of an alkali metal salt of cis-2-alkenoic acid, at elevated temperature, e.g., ≥35° C., for example, ≥40° C., e.g., the gradual addition of the 1,3-dibromo-2-alkanone takes place when the reaction mixture is held at a temperature in the range of 40° C. to 60° C. The molar ratio of 1,3-dibromo-2-alkanone added to the carbonate is from 1:2 to 1:4, e.g., around 1:3-1:3.5.

The use of potassium carbonate, for example, is preferred over sodium carbonate because, as shown below, the corresponding alkali bicarbonate is a by-product of the rearrangement reaction. Less difficulties are likely to be encountered at the work-up stage of the reaction mixture when potassium salts are used, owing to the higher solubility of potassium bicarbonate in water, compared to sodium bicarbonate.

In the presence of an alkali metal salt of the cis-2-alkenoic acid, the reaction takes place during the addition of the 1,3-dibromo-2-alkanone to the alkaline reaction mixture. The occurrence of the reaction is marked by pH drop, (i.e., the initial, strongly alkaline pH of 12-14 drops by at least 2 pH units, e.g., 2-4 pH units, during the addition of the 1,3-dibromo-2-alkanone), and by temperature rise (i.e., ΔT_{reactor}) of ˜5 to 10° C.

In contrast, if the 1,3-dibromo-2-alkanone is added to the alkaline solution in the absence of an alkali metal salt of cis-2-alkenoic acid, then the rearrangement of the 1,3-dibromo-2-alkanone progresses poorly, with said added 1,3-dibromo-2-alkanone accumulating in the reaction vessel. The experimental results shown below indicate that the rearrangement of 1,3-dibromo-2-heptanone, 1,3-dibromo-2-octanone and 1,3-dibromo-2-nonanone did not occur during the addition of the crude 1,3-dibromo-2-alkanone. Only after the addition of the crude 1,3-dibromo-2-alkanone has been completed, the pH started to go down and the T_{R} (reactor temperature) started to go up spontaneously, marking the advance of the reaction. Rearrangement of higher homologues, e.g., 1,3-dibromo-2-decanone and 1,3-dibromo-2-undecanone, is more difficult to advance; and practically no progress can be achieved without the help of a catalytically effective amount of an alkali metal salt of the cis-2-alkenoic acid.

After the slow addition of the crude 1,3-dibromo-2-alkanone has been completed (on a laboratory scale, this may last from 30 to 120 min), the reaction mixture is held under stirring for some time, i.e., a cooking period over a few (1-3) hours, at a temperature in the range from 50 to 55° C., for the reaction to reach completion. A pH drop of ˜0.5-1.5 units is observed during the cooking period. The progress of the reaction can be monitored by pH measurement (a constant pH indicates the end of the reaction) and/or GC analysis of the organic phase (to determine the disappearance of the 1,3-dibromo-2-alkanone, i.e., down to ≤1%, area %).

On completion of the rearrangement reaction, the reaction mixture is cooled to room temperature and separated into aqueous (heavy) and organic (light) phases. The organic phase can be discarded (it contains unreacted brominated isomers which accompanied the 1,3-dibromo-2-alkanone, chiefly 3-bromo-2-alkanone and 3,3-dibromo-2-alkanone; and some condensation by-products formed during the rearrangement reaction). The aqueous phase, which contains the cis-2-alkenoic acid in the form of its alkali metal salt (namely, sodium or potassium salts, determined by the base selected) is worked-up to isolate the product.

One exemplary rearrangement reaction is illustrated by the scheme depicted below, transforming 1,3-dibromo-2-decanone (1,3-DBD) using K₂CO₃ into the potassium salt of cis-2-decenoic acid (abbreviated CDA-K):

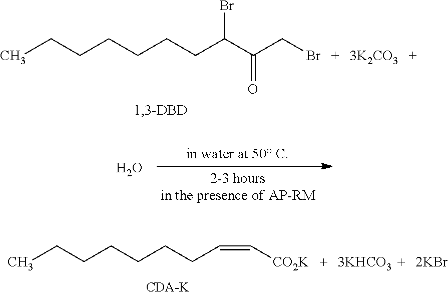

AP-RM indicates the catalytically effective amount of the alkali metal salt of cis-2-alkenoic acid, added in advance to start up the rearrangement reaction. As pointed out above, the catalytically effective amount of the alkali metal salt of the cis-2-alkenoic acid is supplied to the reaction in an aqueous form, for example, by removing a relatively minor portion of the aqueous phase which was collected after the phase separation, and keeping this minor portion for addition in the next run of the process. Usually, the minor portion constitutes from 1 to 10% by weight, e.g., from 3 to 7% (around 5%) of the total weight of the aqueous phase. Based on the concentration of the alkali metal salt of the cis-2-alkenoic acid, it may be appreciated that the catalytically effective amount of the added salt in the alkaline solution before the rearrangement reaction starts is preferably from 1 to 5 molar percent relative to the 1,3-dibromo-2-alkanone.

It should be mentioned, however, that there are alternative ways to supply an alkali metal salt of cis-2-alkenoic acid to the rearrangement reaction, e.g., by the direct addition of the free acid or salt from other sources (if a free acid is added instead of the alkali metal salt, the acid reacts in the alkaline solution to form in situ the corresponding alkali salt).

Next, the major portion of the aqueous phase is worked-up, by washing (repeated washing cycles may be needed) with a water-immiscible organic solvent such as a halogenated hydrocarbon, e.g. dichloromethane, to extract and remove organic impurities from the product-containing aqueous solution.

When the as-obtained reaction mixture cannot be separated into aqueous and organic phases, then it is (optionally) diluted with water and washed with a water-immiscible organic solvent, followed by phase separation, to collect the product-containing, purified aqueous phase, which can be divided into minor and major portions as described above. The minor portion is dedicated to the next run, whereas the major portion is treated to recover the product therefrom.

To recover the product in the form of the free acid, the purified aqueous solution is acidified, e.g., with the aid of concentrated hydrochloric acid (for example, commercially available 32% HCl solution), which is slowly added to the aqueous solution to reach a strongly acidic pH (e.g., from 1 to 2). The acidified reaction mixture is separated into aqueous (heavy) and organic (light) phases. The former contains bromide and chloride salts; the latter consists of the crude cis-2-decenoic acid, and possibly some residual organic solvent which served in the washing stage, and water, which are removed, e.g., by evaporation under vacuum, whereby the crude cis-2-alkenoic acid is obtained.

The sequence of reactions taking place upon acidification of the aqueous solution (specifically, in the preparation of the potassium salt of cis-2-decenoic acid) are shown below:

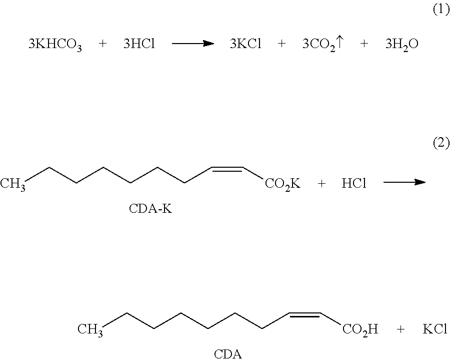

Accordingly, the process of preparing crude cis-2-alkenoic further comprises the acidification of the purified aqueous phase (i.e., after the extraction with the organic solvent) to obtain biphasic medium, comprised of a heavy, salt-containing aqueous phase, and a light organic phase consisting essentially of the cis-2-alkenoic acid in the form of the free acid.

The corresponding alkali salts can be prepared by conventional methods, e.g., by reacting the free acid with potassium hydroxide in a suitable solvent and separating by crystallization and filtration, followed by drying.

As pointed out above, crude cis-2-alkenoic acids afforded by the process of the invention require no further purification, i.e., the acids are pure enough to act as bio-dispersants in bromine-based water treatments, i.e., their purity levels are >80%, >85%, >87%, e.g., from 80 to 95% (by GC, area %). Characteristic purity levels of the crude acids are tabulated in Table B below. However, if needed, the crude acid can be purified by conventional techniques, e.g., chromatography or distillation, or by the method of the invention, which shall now be described in detail.

TABLE B

| | Purity |
|---|---|
| crude cis-2-alkenoic acid | GC, area % |
| 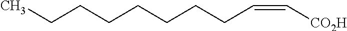 | 89-90 (89.6) |
| (CUDA) | |
| 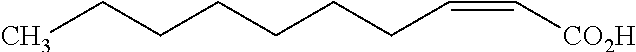 | 87-89 (88.2) |
| (CDA) | |
| 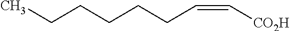 | 91-93 (92.0) |
| (CNA) | |
| 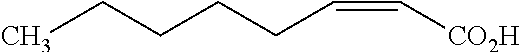 | 88-90 (89.6) |
| (COA) | |
| 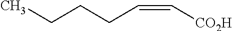 | 94-96 (95.6) |
| (CHA) | |

The purification of crude cis-2-alkenoic acids by several methods was studied. In the study shown below, CDA with HPLC assay of ˜70% (absolute quantification based on calibration with a commercially available external standard (≥97%)) was purified by chromatography and thermal separation techniques such as vacuum distillation and wiped-film evaporation. The goal of the study was to achieve high purity CDA (90% by the HPLC assay mentioned above, or ≥95% HPLC purity by area % (relative)), in conjunction with industrially acceptable yield (60%; 70%; ≥75%; say, from 60 to 80% or from 80 to 90-95%).

Experimental results reported below indicate that very high purity levels are attained by liquid-solid column chromatography, but the yield may often not be satisfactory. For example, with silica gel as adsorbent, using saturated hydrocarbon (heptane or hexane) and an ester (ethyl acetate) as solvents, akin to Preparation 3 of WO 2020/240559, purity levels as high as 99% HPLC % area were reached, but the yield of the final product was quite low (˜25%-40% yield).

On the other hand, purification by thermal separation, e.g., wiped film evaporation (thin film evaporation) was found to be amenable to large scale production as it combines high purity product with an acceptable yield. Despite the thermal lability of cis-2-alkenoic acids, process variables (e.g., reduced pressure, temperature and time) can be adjusted to suppress formation of undesired impurities driven by high temperatures. Assessment of the impurity profile of CDA that is reported below indicates that at high temperatures and reduced pressure, the major impurities consist of the trans isomer of the acid (resulting from isomerization of the desired cis isomer), and bromomethylidene nonanoic acid (present in the crude CDA and originating from intermediate impurity 1,3,3-dibromodecane formed during decanone bromination). The latter is generally the predominant impurity but the trans isomer is not insignificant, and if the evaporation/distillation is not managed properly the amount of the trans isomer can exceed 1.0%. Thus, preparation of a product of high assay (≥90%) with good production yield (60-80%) can be achieved with wiped film evaporation carried out at a temperature of not less than 150° C., for example, in the range from 150 to 180° C. or 150 to 190° C. Isomerization of the cis isomer to the trans isomer is restrained effectively (<3%, e.g., <1%), without showing a drop in product yield, when the pressure is reduced as much as possible (<5 mbar).

For example, effective purification of cis-2-alkenoic acids can be achieved by thermal separation with the aid of a wiped-film evaporator or short path evaporator, operating at feed rates in the range of 14 to 25 ml/min, heating temperature of 150 to 180° C., wherein the evaporator is held at low vacuum down to 2-5 mbar, with evaporator size of 0.12 m². The wetted material is usually made of stainless steel or glass. The thickness of a film formed is usually <1 mm. Vapors are condensed on a condenser and a distillate is collected.

In wiped film evaporation (WFE), feed flow rate is yet another process variable that can be adjusted. For example, in a mini pilot scale with feed flow rates maintained in range of 13-25 ml/min at 150° C. (e.g., 14-18 ml/min; ˜14-15 ml/min) and 32-37 ml/min at 180° C. (e.g., 33-35 ml/min), industrially desired D/F ratio (defined as the ratio between distillate stream leaving the distillation apparatus and feed stream entering the apparatus) of not less than 50%, e.g., >60% is reached. Higher flow feed rates could be employed with larger evaporator size (as shown in the pilot study reported below).

Vacuum pumps that can be employed to give pressure down to <4.5 mbar include an oil pump such as Edwards 2stage oil pump. Pumps to deliver the feed are, for example, peristaltic pump such as a Watson-Marlow 505U pump.

Accordingly, the process of the preparation of cis-2-alkenoic acid as described herein is optionally followed by a purification step comprising thermal separation by wiped film evaporation; e.g., wiped film evaporation of crude cis-2-alkenoic acid while maintaining, during the wiped film evaporation, a temperature-reduced pressure profile whereby the isomerization of the cis-2-alkenoic acid to the trans isomer is suppressed (e.g., less than 1.0% by HPLC area %) while yield is increased (e.g., >60%, >70%, >80%, 90%). For example, a temperature-reduced pressure-time profile is maintained by adjusting the temperature, vacuum and feed flow rates as set out above.

In large scale production a two-stage wiped film evaporation may be beneficial, performing two distillation passes in a single run, e.g., collecting a condensate and returning the condensate to the feed stream, to achieve good results, e.g., evaporation yield of 60%-90%, e.g., 70-908, and high purity (assay by HPLC >90%).

CDA produced by different synthetic methods can benefit from the purification techniques described herein. Thus, another aspect of the invention is a method of purification of cis-2-alkenoic acids, such as CDA, by thermal separation such as wiped film evaporation (employing the conditions set out above) and vacuum distillation.

### IN THE DRAWINGS

FIGS. 1A, 1B and 1C are ¹H-NMR spectra of CDA of Example 1.

FIGS. 2A, 2B and 2C are ¹H-NMR spectra of CDA of Example 2.

FIGS. 3A, 3B and 3C are ¹H-NMR spectra of CUDA of Example 4.

FIGS. 4A, 4B and 4C are ¹H-NMR spectra of CNA of Example 5.

FIGS. 5A, 5B and 5C are ¹H-NMR spectra of COA of Example 6.

FIGS. 6A, 6B and 6C are ¹H-NMR spectra of CHA of Example 7.

FIGS. 7A, 7B and 7C are ¹H-NMR spectra of CDA of Example 8B.

FIGS. 8A and 8B are ¹³C-NMR spectra of CDA of Example 8B.

FIGS. 9A, 9B and 9C are ¹H-NMR spectra of CDA of Example 11.

FIGS. 10A and 10B are ¹³C-NMR spectra of CDA of Example 11.

FIGS. 11A and 11B show isomerization of the cis isomer into the trans isomer, at 150° C. and 180° C., respectively.

### EXAMPLES

### Methods

### GC: Gas-Chromatograph HP 7890A

Method (CDA): Initial temp. 50° C., held 2 min, then raised to 280° C. at 10° C./min and held for 5 min, then raised to 300° C. at 10° C./min and held for 2 min.
Injector: 250° C.
Detector: 300° C.
Split ratio: 1:40
Concentration of the product sample: ˜20 mg/ml DCM
Injection amounts: 1 μl sample
Column: Agilent J&W Columns, HP-5, 30 m×0.32 mm×0.25μ
Part no. 19091J-413, Ser. No. USF302346H

### ¹H-NMR Spectroscopy

Spectra were taken on an Avance III, 500 MHz instrument.

### HPLC: Agilent 1220 LC System

Chromatographic conditions for CDA:
Wavelength: λ=214 nm
Column: Akzo Nobel, Kromasil, 250 mm*4.6 mm ID*5 μm+Pre-Column C18
Mobile phase: [A]H₂O+0.1% H₃PO₄ (conc. 85%):
[B] Acetonitrile (AcN)
Gradient profile:

| Time (min) | H2O (%) | AcN (%) |
|---|---|---|
| 0 | 42 | 58 |
| 28 | 42 | 58 |
| 30 | 5 | 95 |
| 45 | 5 | 95 |
| 46 | 42 | 58 |

Post time: 9 min
Flow: 1.0 ml/min
Injection volume: 5.0 μl
Column temperature: 25° C.
Sample temperature: 20° C.
Retention time of CDA: 14.6 min

### Example 1

### Preparation of Cis-2-Decenoic Acid

### Step 1:

Into a mixture of 2-decanone (200 g, 1.28 mol) and aq. 48% HBr (300 g), stirred and cooled to ˜10° C., was added bromine (410 g, 2.56 mol), dropwise over 2 h. The reaction started immediately with the start of the addition of the bromine and no accumulation of bromine was observed.

The reaction was exothermic and accompanied by the emission of HBr gas, just before the end of the addition of the bromine, which was absorbed in a scrubber.

Most of the reaction took place during the addition of the bromine and cooking at room temperature (˜20° C.) for 6 hours. After standing overnight (˜15 h) at room temperature, without stirring, the composition of the reaction mixture stabilized. Partial conversion of the 3,3-dibromo-2-decanone (3,3-DBD) to the desired product, 1,3-dibromo-2-decanone (1,3-DBD), took place. To the reaction mixture was added water (160 g) at RT, with stirring for 30 min, and the phases were separated.

An aqueous phase (627 g) was obtained containing ˜50% HBr (d=1.51 g/ml) and crude DBD (404 g, d=1.43 g/ml). The concentration of 1,3-DBD in the crude product was 69.6% (GC, area %).

### Step 2:

An aqueous solution of K₂CO₃, in a concentration of 25% w/w, was prepared in a 1 L stirred reactor by the batchwise addition of K₂CO₃ (200 g) to water (600 g). The reaction was exothermic. To this solution was added a part of the aqueous phase (which contained CDA-K) of the reaction mixture (50 g) remaining from a previous run (named AP-RM; see comparative Example 3). The clear solution obtained was heated to 40° C. and crude DBD of step 1 (200 g) was added to it dropwise over 60 min. The progress of the reaction was monitored by GC and by the change in the pH. The reaction was completed by cooking at 50° C. for 3.0 h, with mechanical stirring.

It should be pointed out that without the addition of AP-RM, the reaction only starts spontaneously two hours after the addition of the crude DBD.

The end of the reaction was determined by the pH (drop in the pH from 13.3 to 9.3) and by GC analysis of the reaction mixture (disappearance of 1,3-DBD to <1%, area %). After completion of the reaction, cooling to RT and stopping the stirring, an organic phase appeared above the aqueous phase which contained unreacted 3-bromo-2-decanone (3-BD) and 3,3-DBD, and by-products formed by a condensation reaction of crude DBD. The phases were separated. The organic phase (39 g) was organic waste. 50 g of the aq. phase was taken for use in the next run.

In order to reduce the amount of impurities to a minimum, the remainder of the aqueous phase (950 g) was washed three times with dichloromethane (DCM, 3×250 g).

After the washing stage, an aqueous phase was obtained containing cis-2-decenoic acid potassium salt (CDA-K), organic by-products, KBr and KHCO₃. In order to obtain the crude cis-2-decenoic acid (CDA), the aqueous phase was acidified by the dropwise addition of aq. 32% HCl (193 g) over 1 h. During the acidification (final pH=1.1), CO₂ (calculated at 63 g) was emitted.

After stopping the stirring, an aqueous phase (955 g) was obtained containing salts: KCl and KBr (heavy phase, d=1.19 g/ml) and wet crude CDA (light phase, 71 g, d=1.07 g/ml).

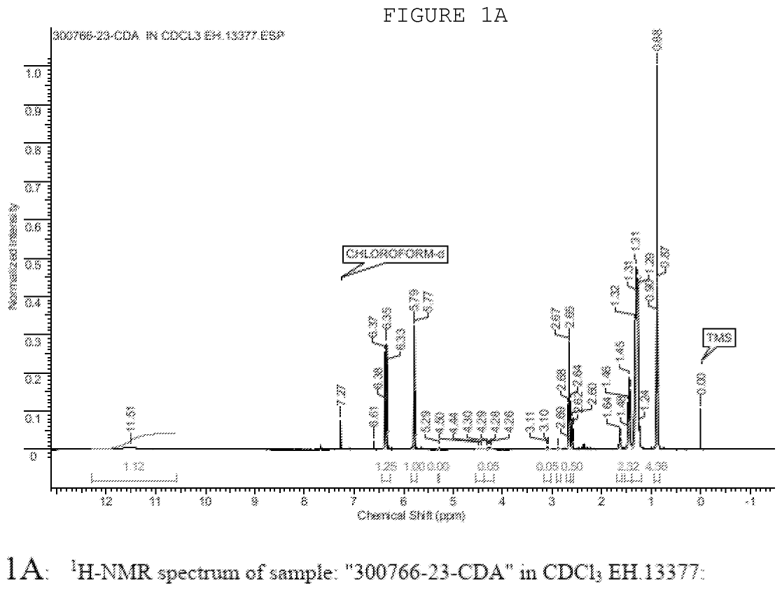

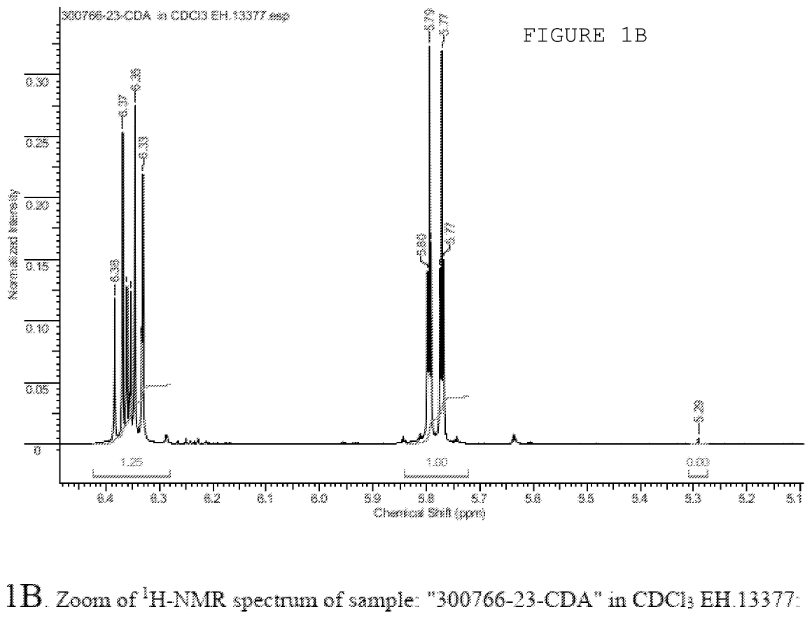

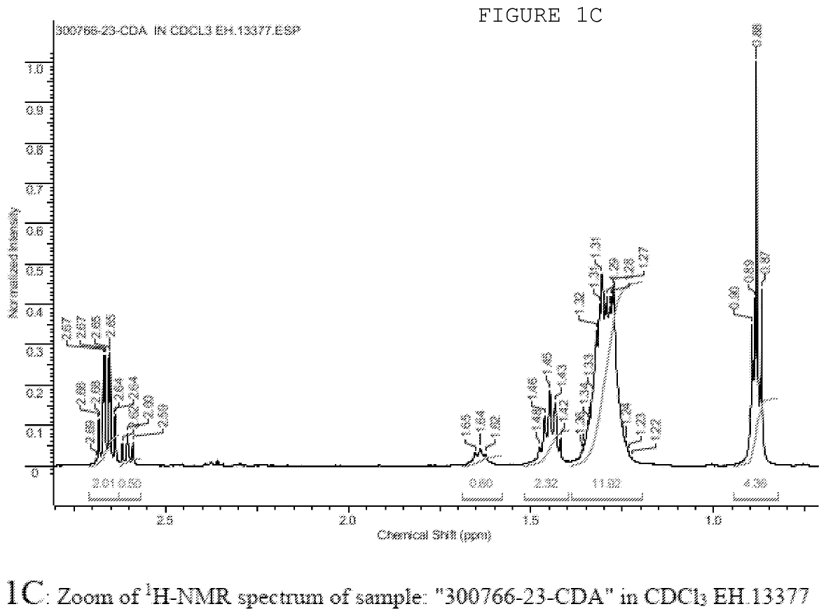

Evaporation of the DCM and lights from the wet CDA under vacuum (at T_{B}=50° C.) gave crude CDA (50.5 g), which was analysed by GC and ¹H-NMR (see FIGS. 1A, 1B and 1C for 1H-NMR spectra). The calculated yield of crude CDA was ˜68%, based on 1,3-DBD, or 46.8%, based on 2-decanone.

The purity of the crude CDA obtained was 88.2% (by GC area %). The main impurity in the crude product was 2-bromomethylidene nonanoic acid (BMNA): 8.8% (by GC, area %).

### Example 2

### Preparation of Cis-2-Decenoic Acid

### Step 1:

Into a mixture of 2-decanone (400 g, 2.564 mol) and aq. 48% HBr (600 g), stirred and cooled to ˜10° C., was added bromine (800 g, 5 mol), dropwise over 5 h. The reaction started immediately with the start of the addition of the bromine and no accumulation of bromine was observed. The reaction was exothermic and accompanied by the emission of HBr gas, just before the end of the addition of the bromine, which was absorbed in a scrubber.

Most of the reaction took place during the addition of the bromine, and after standing overnight at room temperature, without stirring, the composition of the reaction mixture stabilized. Partial conversion of the 3,3-dibromo-2-decanone (3,3-DBD) to the desired product, 1,3-dibromo-2-decanone (1,3-DBD), took place. To the reaction mixture was added water (300 g) at RT, with stirring for 30 min, and the phases were separated.

An aqueous phase (1207 g) was obtained containing ˜49.5% HBr (d=1.51 g/ml) and crude DBD (789 g, d=1.42 g/ml). The concentration of 1,3-DBD in the crude product was 70.4% (GC, area %).

### Step 2:

An aqueous solution of K₂CO₃, in a concentration of 25% w/w, was prepared in a 2 L stirred reactor by the batchwise addition of K₂CO₃ (400 g) to water (1200 g). The reaction was exothermic. To this solution was added a part of the aqueous phase of the reaction mixture of CDA-K (50 g) remaining from a previous run. The clear solution obtained was heated to 40° C. and crude DBD (Step 1, 400 g) was added to it dropwise over 70 min. The progress of the reaction was monitored by GC and by the change in the pH. The reaction was completed by cooking at 40° C. for 1.0 h, then at 50° C. for 2.0 h, with mechanical stirring.

The end of the reaction was determined by the pH (drop in the pH from 12.7 to 9.6) and by GC analysis of the reaction mixture (disappearance of 1,3-DBD to <1%, area %). After completion of the reaction, cooling to RT and stopping the stirring, an organic phase appeared above the aqueous phase which contained unreacted 3-BD and 3,3-DBD, and by-products formed by a condensation reaction of crude DBD. The phases were separated. 50 g of the aq. phase was taken for use in the next run.

In order to reduce the amount of impurities to a minimum, the aqueous phase (1943 g) was washed three times with dichloromethane (DCM, 3×500 g).

After the washing stage, an aqueous phase was obtained containing cis-2-decenoic acid potassium salt (CDA-K), organic by-products, KBr and KHCO₃. In order to obtain the crude cis-2-decanoic acid (CDA), the aqueous phase was acidified by the dropwise addition of aq. 32% HCl (401 g) over 1 h. During the acidification (final pH=1.9), CO₂ (calculated at 127 g) was emitted.

After stopping the stirring, an aqueous phase (2017 g) was obtained containing salts: KCl and KBr (heavy phase, d=1.19 g/ml) and wet crude CDA (light phase, 128 g, d=1.03 g/ml).

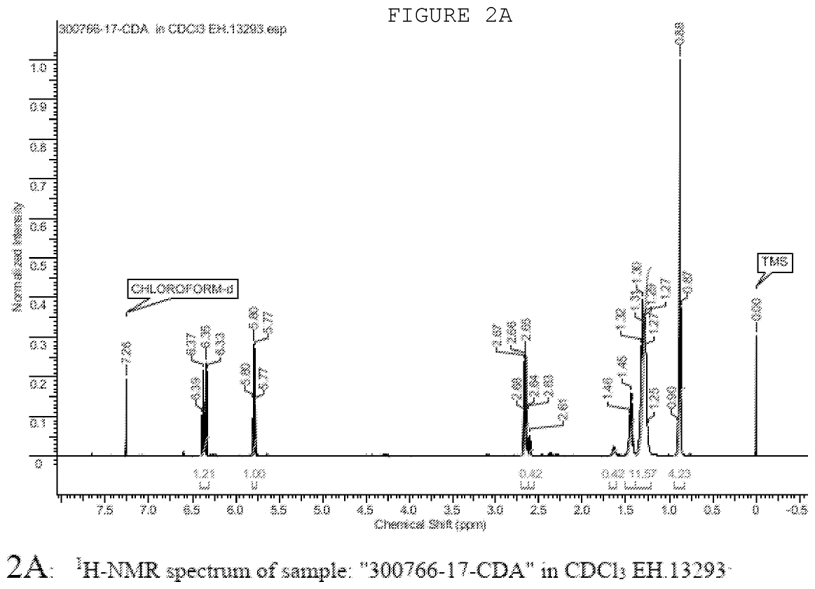

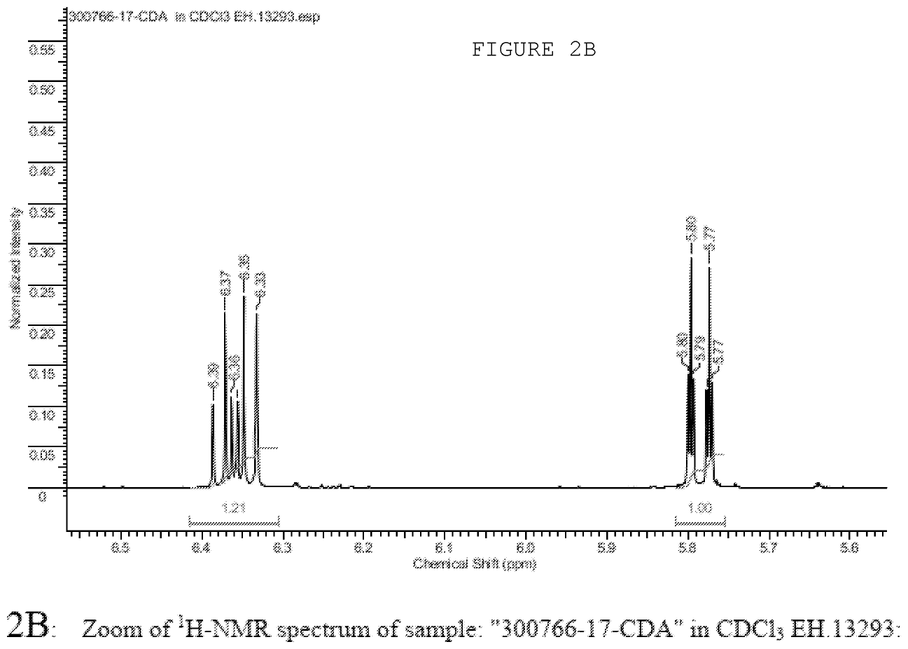

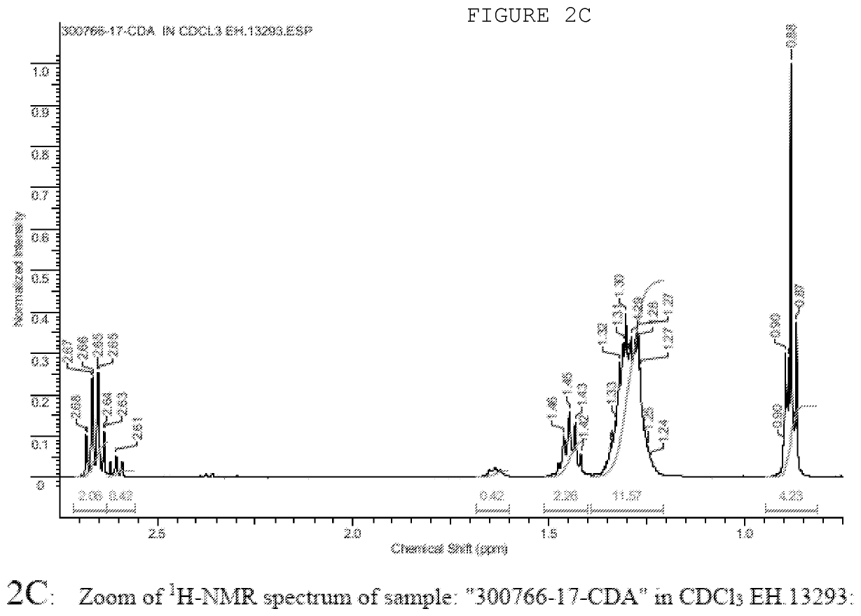

Evaporation of the DCM and lights from the wet CDA under vacuum (T_{B}=50° C.) gave crude CDA (102 g), which was analyzed by GC, HPLC and ¹H-NMR (¹H-NMR spectra in FIGS. 2A, 2B and 2C).

Based on the results, the purity of the crude CDA obtained was 89.7% (by GC area %) and 90.0% (by HPLC, area %). The calculated yield of crude CDA was ˜67% based on 1,3-DBD.

### Example 3 (Comparative)

### Preparation of Cis-2-Decenoic Acid

Step 1 was carried out as in Example 1. The rearrangement reaction of Step 2, however, was carried out without the addition of the alkali metal salt of cis-2-alkenoic acid.

### Step 2:

An aqueous solution of K₂CO₃, in a concentration of 25% w/w, was prepared in a 1 L stirred reactor by the batchwise addition of K₂CO₃ (200 g) to water (600 g). The reaction was exothermic. The clear solution obtained was heated to 40° C. and crude DBD (200 g; obtained as previously described) was added to it dropwise over 60 min. The progress of the reaction was monitored by GC and by the change in the pH.

The mixture of aq. K₂CO₃ and crude DBD was stirred for 3 h at a temperature of 50° C. Based on the pH (unchanged at ˜13) and on GC, it was seen that no reaction had taken place.

Then suddenly, the temperature in the reactor started to rise spontaneously and reached 76° C. within ten minutes. The end of the reaction was determined by the pH (drop in the pH from 13.3 to 9.5) and by GC analysis of the reaction mixture (disappearance of 1,3-DBD to <1%, area %). The phases were separated, and 50 g of the aqueous phase was taken for use in the next run (i.e., the procedure of Example 1).

### Example 4

### Preparation of Cis-2-Undecenoic Acid

### Step 1:

Into a mixture of 2-undecanone (218 g, 1.28 mol) and aq. 48% HBr (300 g), stirred and cooled to ˜10° C., was added bromine (410 g, 2.56 mol), dropwise over 3 h. The reaction started immediately with the start of the addition of the bromine and no accumulation of bromine was observed. The reaction was exothermic and accompanied by the emission of HBr gas, just before the end of the addition of the bromine, which was absorbed in a scrubber.

Most of the reaction took place during the addition of the bromine and cooking at room temperature (˜20° C.) for 3.5 hours. After standing overnight (˜16.5 h) at room temperature, without stirring, the composition of the reaction mixture stabilized. Partial conversion of the 3,3-dibromo-2-undecanone (3,3-DBUD) to the desired product, 1,3-dibromo-2-undecanone (1,3-DBUD), took place. To the reaction mixture was added water (160 g) at RT, with stirring for 30 min, and the phases were separated.

An aqueous phase (627 g) was obtained containing ˜50% HBr (d=1.50 g/ml) and crude DBUD (415 g, d=1.39 g/ml). The concentration of 1,3-DBUD in the crude product was 69.1% (GC, area %).

### Step 2:

An aqueous solution of K₂CO₃, in a concentration of 25% w/w, was prepared in a 1 L stirred reactor by the batchwise addition of K₂CO₃ (200 g) to water (600 g). The reaction was exothermic. The clear solution obtained was heated to 40° C. and crude DBUD of Step 1 (200 g) was added to it dropwise over 20 min. The progress of the reaction was monitored by GC and by the change in the pH.

The mixture of aq. K₂CO₃ and crude DBUD was stirred for 1 h at a temperature of 50° C., for 1.5 h at a temperature of 60° C., and for an additional 1.5 h at a temperature of 70° C. Based on the pH (unchanged at ˜13) and on GC, it was seen that no reaction had taken place.

Next, to the reaction mixture was added a part of the aqueous phase of the reaction mixture of CDA-K (˜10 g) dropwise over 15 min. At the end of the addition, the temperature in the reactor started to rise and reached 82° C. within 10 min. This mixture was then stirred for an additional 2 h at 70° C.

The end of the reaction was determined by the pH (drop in the pH from 13 to 10) and by GC analysis of the reaction mixture (disappearance of 1,3-DBUD to <1%, area %).

In order to reduce the amount of impurities to a minimum, the reaction mixture (960 g) was washed three times with dichloromethane (DCM, 3×250 g) at RT. It should be mentioned that the first phase separation was slow.

After the washing stage, an aqueous phase was obtained containing cis-2-undecenoic acid potassium salt (CUDA-K), organic by-products, KBr and KHCO₃. In order to obtain the crude cis-2-undecenoic acid (CUDA), the aqueous phase was acidified by the dropwise addition of aq. 32% HCl (132 g) over 1 h. During the acidification, CO₂ was emitted.

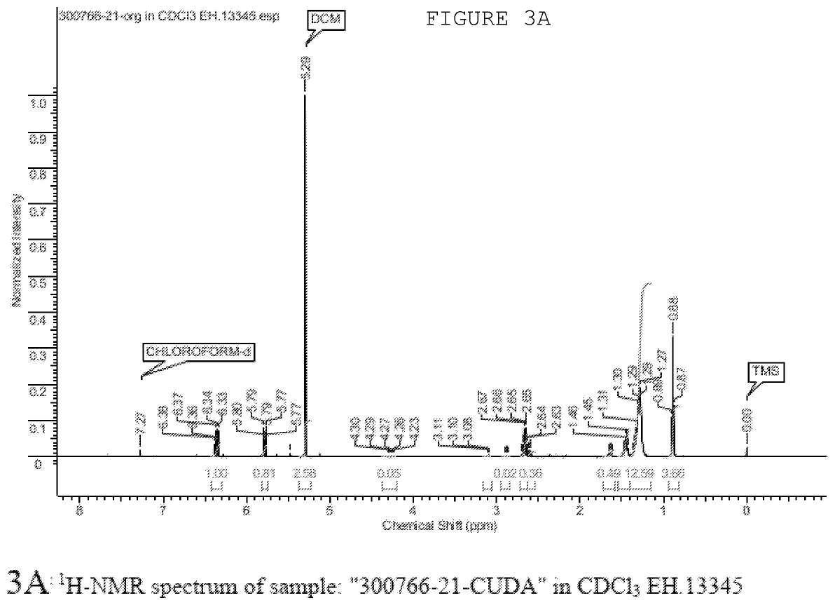

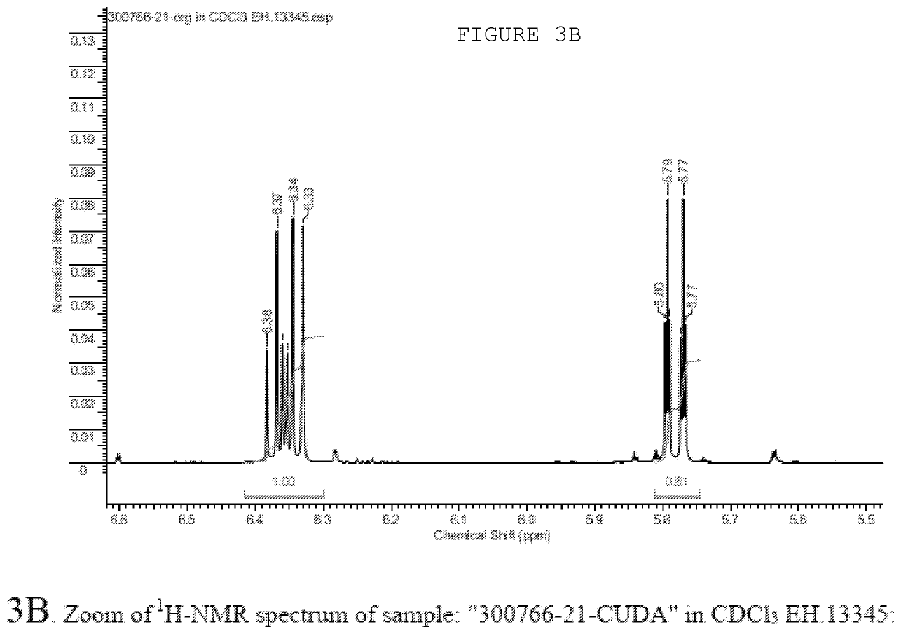

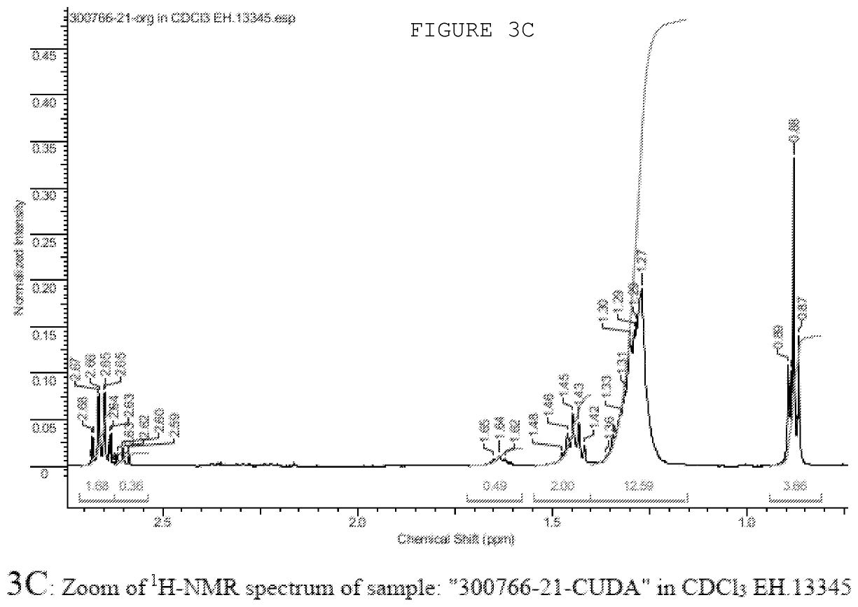

After stopping the stirring, an aqueous phase (762 g) was obtained containing salts: KCl and KBr (heavy phase, d=1.15 g/ml) and wet crude CUDA (light phase, 53 g, d=1.07 g/ml) which was analysed by GC and ¹H-NMR (see FIGS. 3A, 3B and 3C for ¹H-NMR spectra). The purity of the crude CUDA obtained was 89.6% (by GC area %). The main impurity in the crude product was 2-bromomethylidene decanoic acid (BMDA): 5.2% (by GC, area %).

Evaporation of the DCM and lights from the wet CUDA under vacuum (at T_{B}=50° C.) gave crude CUDA (35 g).

It is seen that in this Example, a small amount of alkali metal salt of a homologue acid (CDA-K) was used to advance the preparation of CUDA. The aqueous phase obtained containing cis-2-undecenoic acid potassium salt (CUDA-K), with an insignificant amount of CDA-K, can be used to supply, for the next run, a catalytically effective amount of CUDA-K to be added to the alkaline K₂CO₃ solution before the slow addition of the crude DBUD starts, to ensure an efficient, manageable reaction.

### Example 5 (Comparative)

### Preparation of Cis-2-Nonenoic Acid

### Step 1:

Into a mixture of 2-nonanone (from Sigma-Aldrich; 182 g, 1.28 mol) and aq. 48% HBr (300 g), stirred and cooled to ˜10° C., was added bromine (410 g, 2.56 mol), dropwise over 3 h. The reaction started immediately with the start of the addition of the bromine and no accumulation of bromine was observed. The reaction was exothermic and accompanied by the emission of HBr gas, just before the end of the addition of the bromine, which was absorbed in a scrubber.

Most of the reaction took place during the addition of the bromine and cooking at room temperature (˜20° C.) for 2.0 hours. After leaving overnight (˜17 h) at room temperature, with stirring, the composition of the reaction mixture stabilized. Partial conversion of the 3,3-dibromo-2-nonanone (3,3-DBN) to the desired product, 1,3-dibromo-2-nonanone (1,3-DBN), took place. To the reaction mixture was added water (160 g) at RT, with stirring for 30 min, and the phases were separated.

An aqueous phase (624 g) was obtained containing ˜50% HBr (d=1.50 g/ml) and crude DBN (382 g, d=1.47 g/ml). The concentration of 1,3-DBN in the crude product was 70.6% (GC, area %).

### Step 2:

An aqueous solution of K₂CO₃, in a concentration of 25% w/w, was prepared in a 1 L stirred reactor by the batchwise addition of K₂CO₃ (200 g) to water (600 g). The reaction was exothermic. The clear solution obtained was heated to 46° C. and crude DBN from Step 1 (191 g) was added to it dropwise over 45 min. The progress of the reaction was monitored by the change in the pH and the T_{R}.

Based on the pH (unchanged at ˜13) and on GC, it was seen that no reaction had taken place during the addition of the crude DBN. Immediately after the addition of the crude DBN, the pH started to go down and the T_{R} started to go up.

The end of the reaction was determined by the pH (drop in the pH from 13.3 to 9.1) and by GC analysis of the reaction mixture (disappearance of 1,3-DBN to <1%, area %). The phases were separated. The organic phase (42.6 g) was organic waste.

In order to reduce the amount of impurities to a minimum, the aqueous phase (948 g) was washed three times with dichloromethane (DCM, 3×250 g).

After the washing stage, an aqueous phase was obtained containing cis-2-nonenoic acid potassium salt (CNA-K), organic by-products, KBr and KHCO₃. In order to obtain the crude cis-2-nonenoic acid (CNA), the aqueous phase was acidified by the dropwise addition of aq. 32% HCl (227 g) over 1 h. During the acidification, CO₂ was emitted.

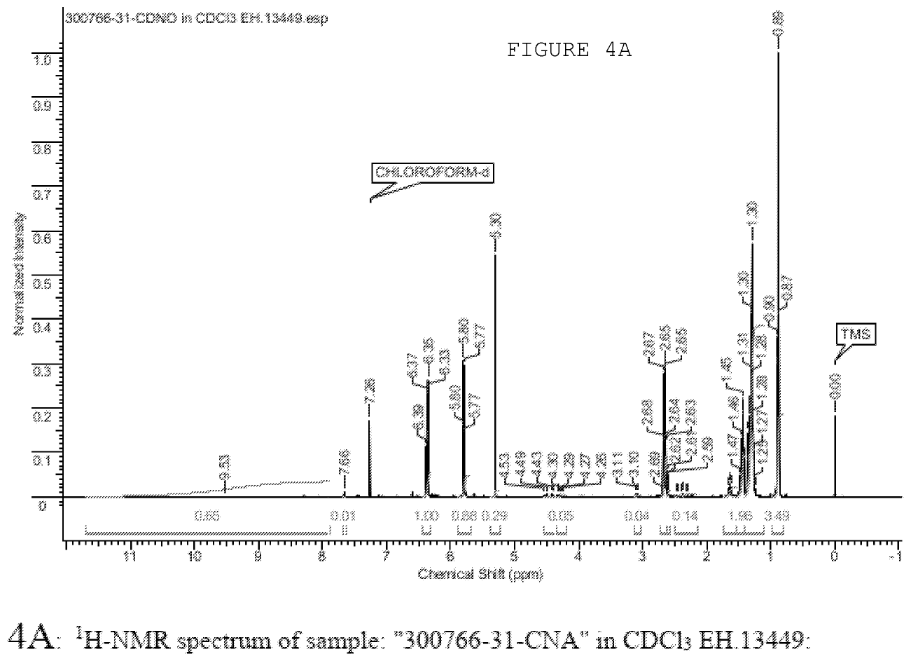

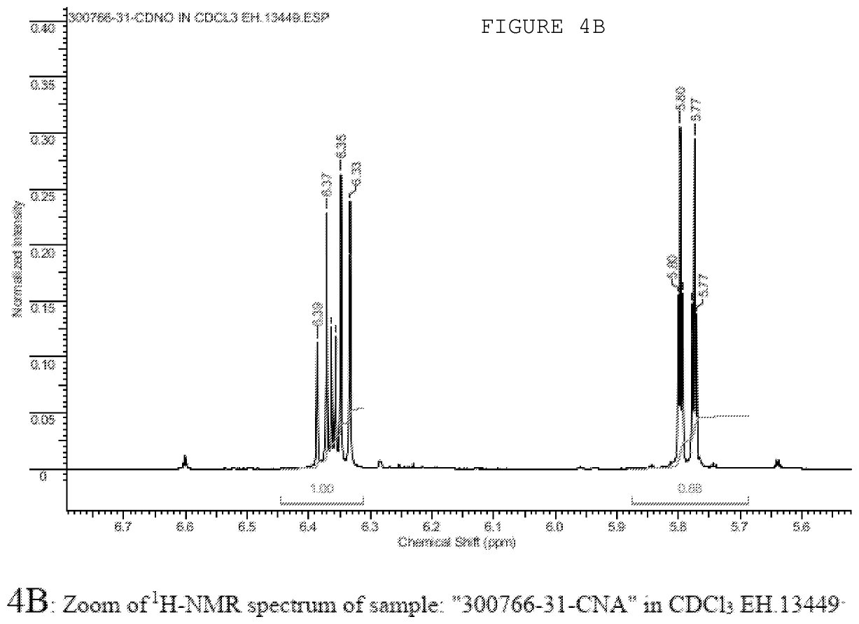

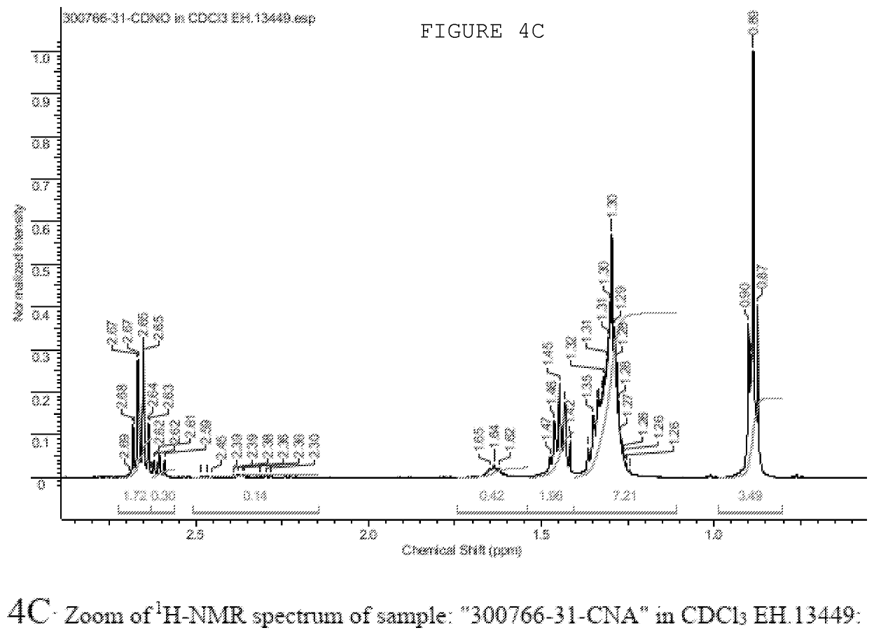

After stopping the stirring, an aqueous phase (978 g) was obtained containing salts: KCl and KBr (heavy phase, d=1.19 g/ml) and wet crude CNA (light phase, 51 g, d=1.02 g/ml) which was analysed by GC and ¹H-NMR (see FIGS. 4A, 4B and 4C for ¹H-NMR spectra). The purity of the obtained CNA was 92.0% (by GC, area %).

Evaporation of the DCM and lights from the wet CNA under vacuum (at T_{B}=50° C.) gave crude CNA (46.6 g).

### Example 6 (Comparative)

### Preparation of Cis-2-Octenoic Acid

### Step 1:

Into a mixture of 2-octanone (from Sigma-Aldrich; 164 g, 1.28 mol) and aq. 48% HBr (300 g), stirred and cooled to ˜10° C., was added bromine (410 g, 2.56 mol), dropwise over 3 h. The reaction started immediately with the start of the addition of the bromine and no accumulation of bromine was observed. The reaction was exothermic and accompanied by the emission of HBr gas, just before the end of the addition of the bromine, which was absorbed in a scrubber.

Most of the reaction took place during the addition of the bromine and cooking at room temperature (˜20° C.) for 2.5 hours. After leaving overnight (˜15 h) at room temperature, with stirring, the composition of the reaction mixture stabilized. Partial conversion of the 3,3-dibromo-2-octanone (3,3-DBO) to the desired product, 1,3-dibromo-2-octanone (1,3-DBO), took place. To the reaction mixture was added water (160 g) at RT, with stirring for 30 min, and the phases were separated.

An aqueous phase (636 g) was obtained containing ˜50% HBr (d=1.51 g/ml) and crude DBO (358 g, d=1.54 g/ml). The concentration of 1,3-DBO in the crude product was 71.0% (GC, area %).

### Step 2:

An aqueous solution of K₂CO₃, in a concentration of 25% w/w, was prepared in a 1 L stirred reactor by the batchwise addition of K₂CO₃ (200 g) to water (600 g). The reaction was exothermic. The clear solution obtained was heated to 49° C. and crude DBO from Step 1 (182 g) was added to it dropwise over 1 h. The progress of the reaction was monitored by the change in the pH and the T_{R}.

Based on the pH (unchanged at ˜13) and on GC, it was seen that no reaction had taken place during the addition of the crude DBO. Immediately after the addition of the crude DBO, the pH started to go down and the T_{R} started to go up.

The end of the reaction was determined by the pH (drop in the pH from 13.7 to 9.3) and by GC analysis of the reaction mixture (disappearance of 1,3-DBO to <1%, area %).

Before starting the washings, water (75 g) was added to the reaction mixture (982 g). In order to reduce the amount of impurities to a minimum, the reaction mixture was washed four times with dichloromethane (DCM, 4×250 g).

After the washing stage, an aqueous phase was obtained containing cis-2-octenoic acid potassium salt (COA-K), organic by-products, KBr and KHCO₃. In order to obtain the crude cis-2-octenoic acid (COA), the aqueous phase was acidified by the dropwise addition of aq. 32% HCl (178 g) over 1 h. During the acidification, CO₂ was emitted.

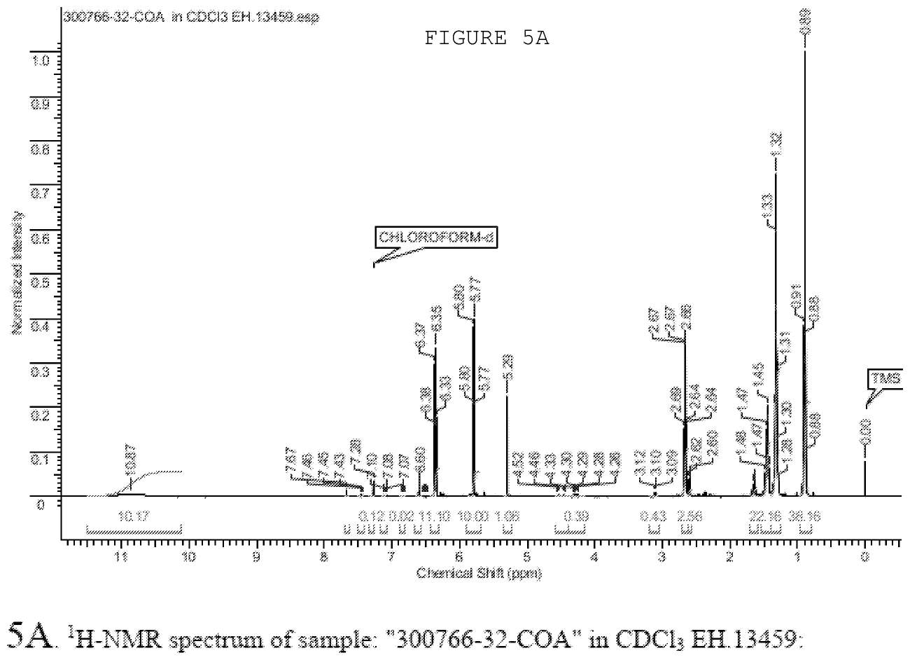

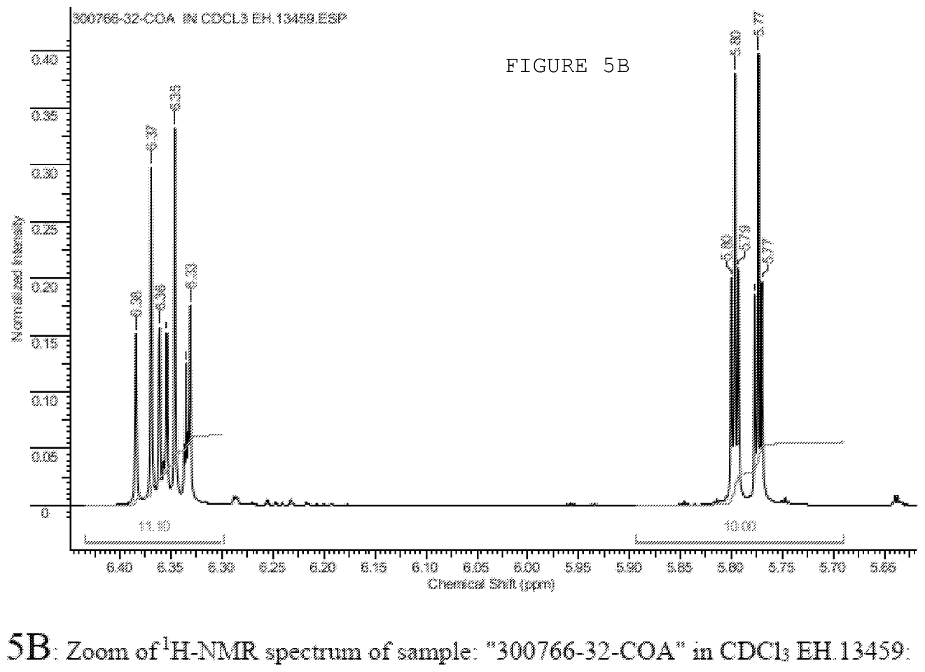

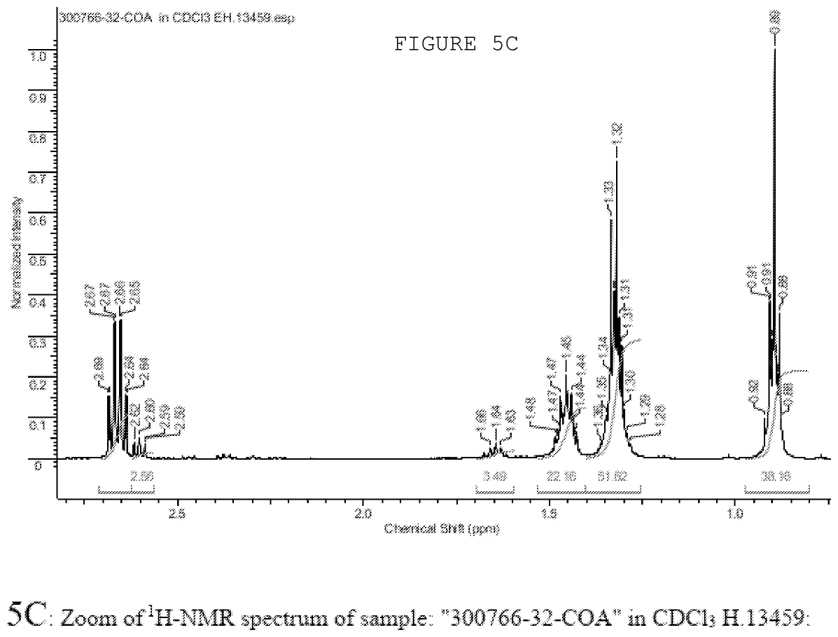

After stopping the stirring, an aqueous phase (938 g) was obtained containing salts: KCl and KBr (heavy phase, d=1.18 g/ml) and wet crude COA (light phase, 44 g, d=1.00 g/ml) which was analysed by GC and ¹H-NMR (see FIGS. 5A, 5B and 5C for ¹H-NMR spectra). The purity of the COA obtained was 89.6% (by GC, area %).

Evaporation of the DCM and lights from the wet COA under vacuum (at T_{B}=50° C.) gave crude COA (41.3 g).

### Example 7 (Comparative)

### Preparation of Cis-2-Heptenoic Acid

### Step 1:

Into a mixture of 2-heptanone (from Sigma-Aldrich; 146 g, 1.28 mol) and aq. 48% HBr (300 g), stirred and cooled to ˜10° C., was added bromine (410 g, 2.56 mol), dropwise over 3 h. The reaction started immediately with the start of the addition of the bromine and no accumulation of bromine was observed. The reaction was exothermic and accompanied by the emission of HBr gas, just before the end of the addition of the bromine, which was absorbed in a scrubber.

Most of the reaction took place during the addition of the bromine and cooking at room temperature (˜20° C.) for 4.5 hours. After standing overnight (˜17 h) at room temperature, with stirring, the composition of the reaction mixture stabilized. Partial conversion of the 3,3-dibromo-2-heptanone (3,3-DBH) to the desired product, 1,3-dibromo-2-heptanone (1,3-DBH), took place. To the reaction mixture was added water (160 g) at RT, with stirring for 30 min, and the phases were separated.

An aqueous phase (631 g) was obtained containing ˜50% HBr (d=1.52 g/ml) and crude DBH (351 g, d=1.60 g/ml). The concentration of 1,3-DBH in the crude product was 72.6% (GC, area %).

### Step 2:

An aqueous solution of K₂CO₃, in a concentration of 25% w/w, was prepared in a 1 L stirred reactor by the batchwise addition of K₂CO₃ (200 g) to water (600 g). The reaction was exothermic. The clear solution obtained was heated to 49° C. and crude DBH from Step 1 (173 g) was added to it dropwise over 1 h. The progress of the reaction was monitored by the change in the pH and the T_{R}.

Based on the pH (unchanged at ˜13), it was seen that no reaction had taken place during the addition of the crude DBH. Immediately after the addition of the crude DBH, the pH started to go down and the T_{R} started to go up.

The end of the reaction was determined by the pH (drop in the pH from 13.5 to 9.3) and by GC analysis of the reaction mixture (disappearance of 1,3-DBH to <1%, area %). After completion of the reaction, cooling to RT and stopping the stirring, an organic phase appeared above the aqueous phase which contained unreacted 3-BH and 3,3-DBH, and by-products formed by a condensation reaction of crude DBH. The phases were separated. The organic phase (24 g) is organic waste.

Before starting the washings, water (50 g) was added to the reaction mixture (948 g). In order to reduce the amount of impurities to a minimum, the diluted reaction mixture (998 g) was washed three times with dichloromethane (DCM, 3×250 g).

After the washing stage, an aqueous phase was obtained containing cis-2-heptenoic acid potassium salt (CHA-K), organic by-products, KBr and KHCO₃. In order to obtain the crude cis-2-heptenoic acid (CHA), the aqueous phase was acidified by the dropwise addition of aq. 32% HCl (193 g) over 1 h. During the acidification, CO₂ was emitted.

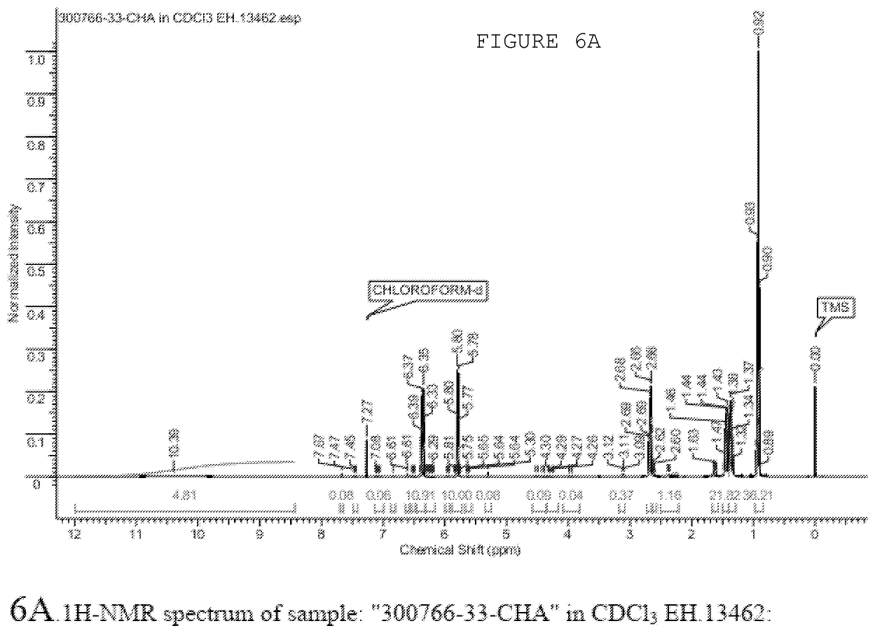

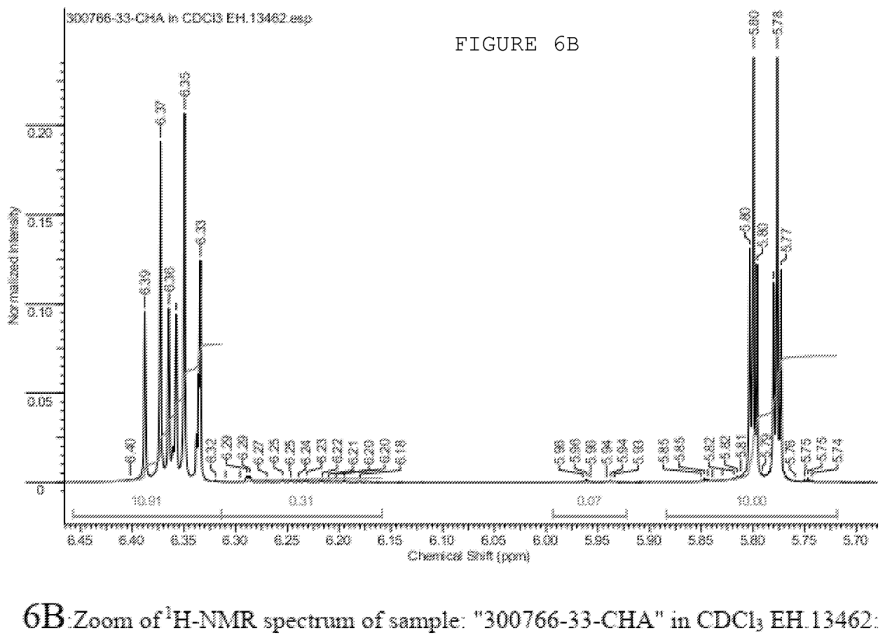

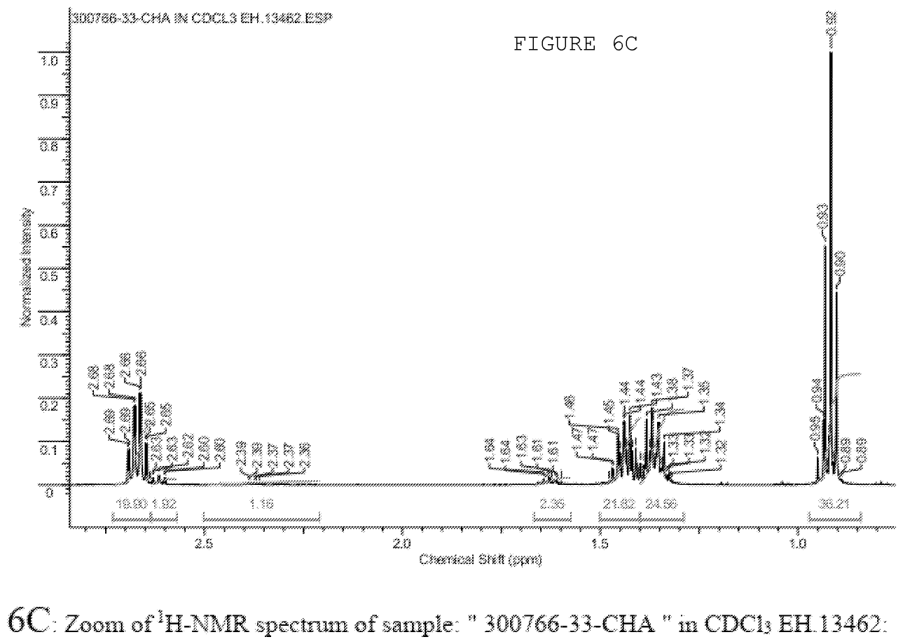

After stopping the stirring, an aqueous phase (1014 g) was obtained containing salts: KCl and KBr (heavy phase, d=1.18 g/ml) and wet crude CHA (light phase, 45 g, d=1.00 g/ml) which was analysed by GC and ¹H-NMR (see FIGS. 6A, 6B and 6C for ¹H-NMR spectra). The purity of the CHA obtained was 95.6% (by GC, area %).

Evaporation of the DCM and lights from the wet CHA under vacuum (at T_{B}=50° C.) gave crude CHA (44 g).

### Examples 8A-8B (Comparative)

### Purification of Crude Cis-2-Decenoic Acid by Silica Gel Column Chromatography

### 8A

1.54 g of crude CDA was placed on top of a 24 cm ø22 mm diameter glass column packed with 75 mL silica-gel 60 (Merck 0.04-0.063 mm). The initial eluent was hexane and a head of 240 mL eluent was discarded. Then the eluent changed to 5% ethyl acetate-95% hexane. An extra 140 mL eluent was discarded then a 60 mL fraction was collected. The solvents were removed under reduced pressure affording 0.36 g of pure CDA (99% by HPLC % area).

### 8B

2.35 g of crude CDA was placed on top of a 35 cm 022_mm diameter glass column packed with 100 mL silica-gel 60 (Merck 0.04-0.063 mm). The initial eluent was hexane and a head of 300 mL eluent was discarded. Then the eluent was changed to 5% ethyl acetate-95% hexane. An extra 210 mL eluent was discarded then a 65 mL fraction was collected.

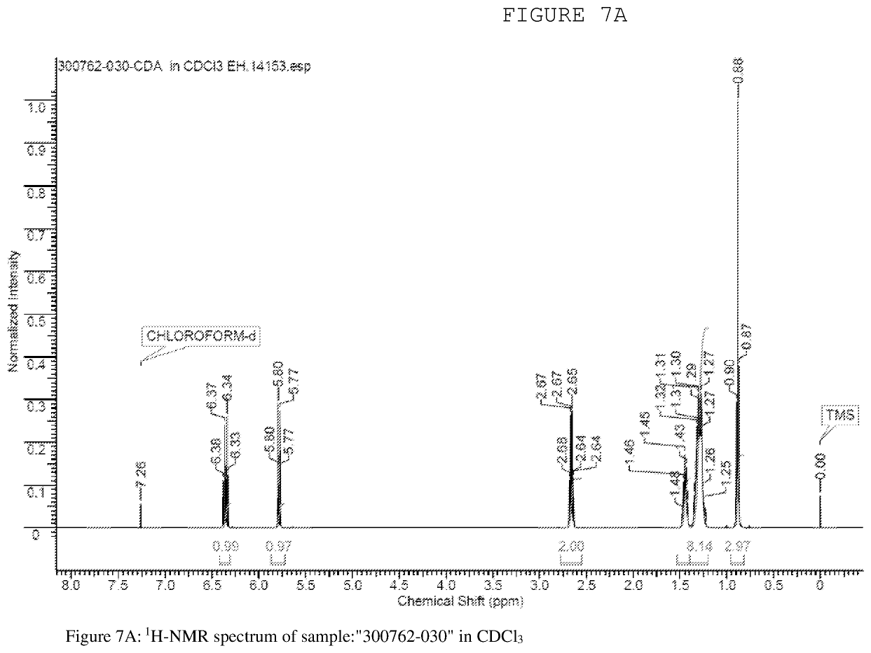

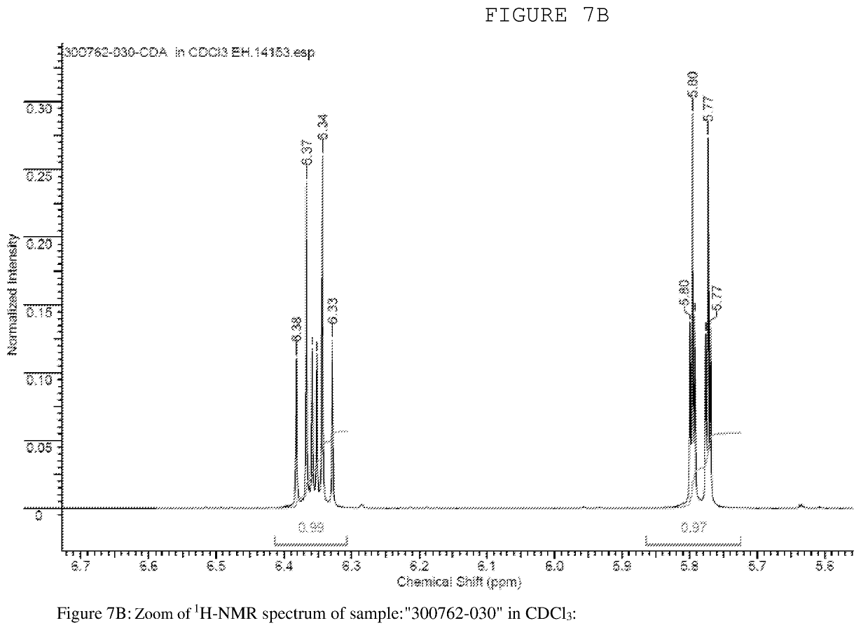

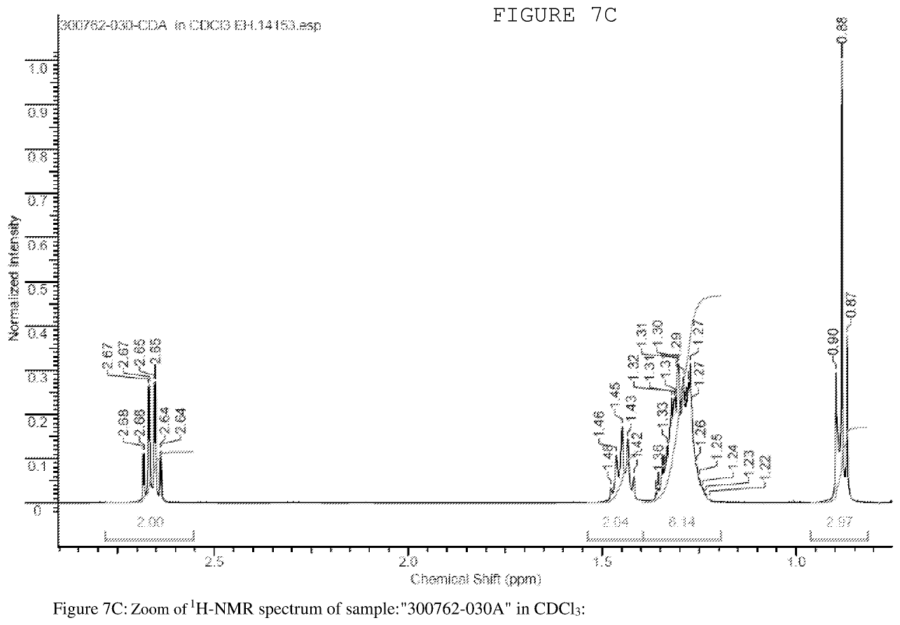

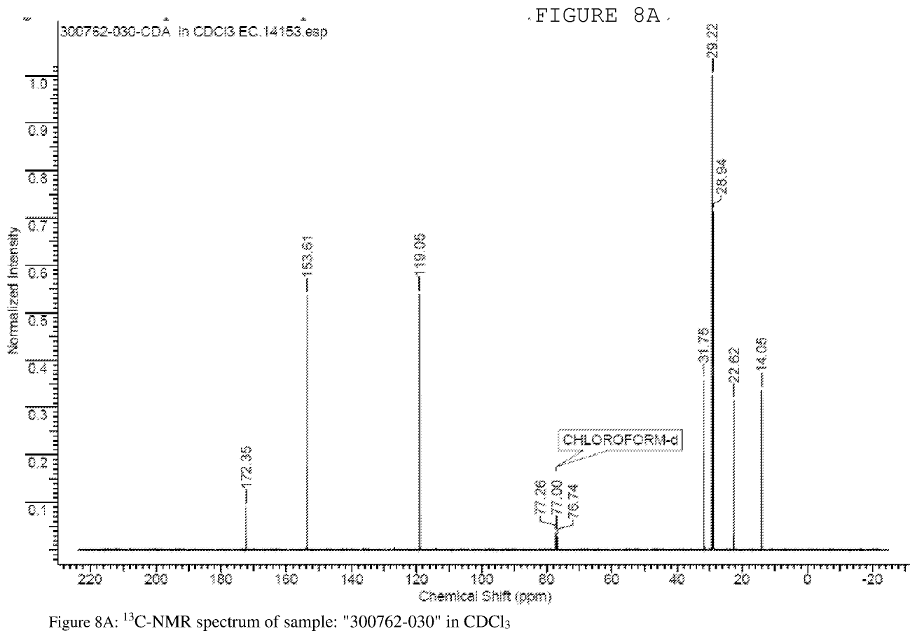

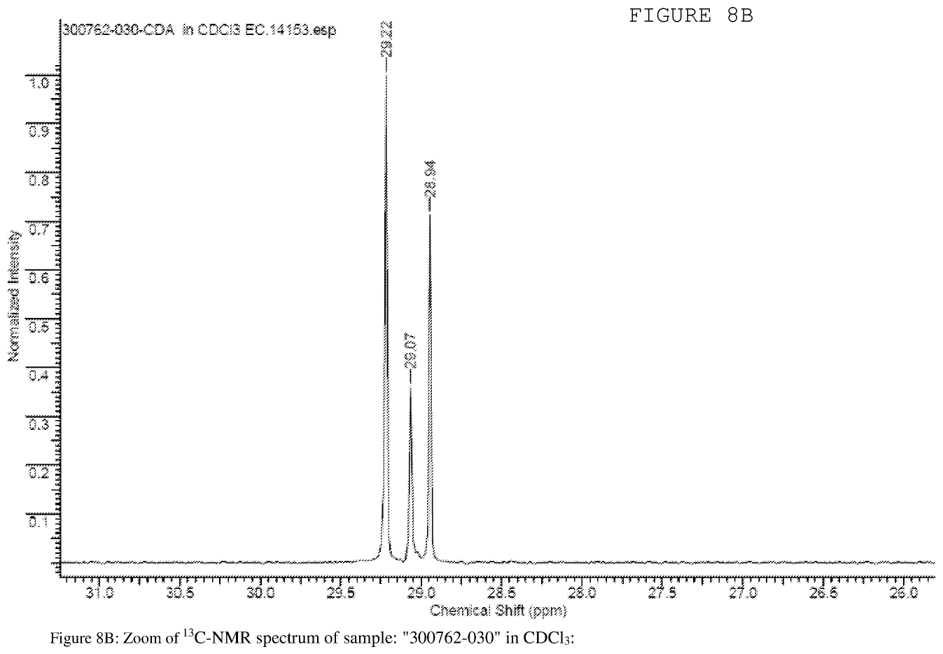

This fraction was combined with the fraction of 8A and solvents were removed under reduced pressure affording 0.95 g of pure CDA (99% by HPLC % area) which was analyzed by GC and ¹H-NMR (see FIGS. 7A, 7B and 7C for ¹H-NMR spectra and FIGS. 8A and 8B for ¹³C-NMR).

### Example 9

### Purification of Crude Cis-2-Decenoic Acid by Distillation (Laboratory Scale)

Crude CDA (CDA content 70% by HPLC assay) was distilled under reduced pressure. The distillation apparatus consisted of a 100 mL three-neck flask connected to a short Vigreux column through a Y connector. A water-cooled condenser and four-flasks rotating receiver were attached to the vacuum pump (2 mbar). Crude CDA (79.7 g) was placed in the distillation flask, vacuum and heating were applied. Distillation started at a 77-83° C. vapor temperature at the distillation head; pure CDA was obtained at a vapor temperature of 95-105° C. (bottom temp 130-140° C.). 43 g of CDA was collected, with CDA content of 96.7% (assay by HPLC). The distilled CDA was 97.6% pure by GC (% area), accompanied by 0.6% trans-decenoic acid and ca. 0.5% bromomethylidene nonanoic acid. The distillation yield was 75%.

### Example 10

### Purification of Crude Cis-2-Decenoic Acid by Wiped-Film Evaporation (Mini Pilot-Scale)

A mini pilot scale study was performed to determine the evaporation conditions that achieve purification of crude CDA to reach a high CDA assay (90%) alongside good yields (>90%).

For CDA purification a glass WFE 0.12 m² was used, a 1 L bottom flask for residue and a 200 ml flask for distillate collection. The heating was performed by thermal oil circulated by a LAUDA system to the WFE jacket. The vapors were passed through the condenser, and cooled by chilled 7° C. ethylene glycol-water. Crude CDA was dosed to the WFE by a peristaltic pump (over ½″ Teflon tube). In order to monitor the feed and distillation rate during the performed runs, lab measuring cylinders were used for the crude inlet and distillate outlet. A vacuum was reached by an oil vacuum pump, accompanied by an acetone-dry ice trap to prevent DCM from reaching the pump.

A series of runs were performed, varying the conditions (temperature, diminished pressure, feed flow rate, rotation speed, D/F ratio) and testing their influence on the assay and impurities profile. Operation conditions and results of tests are tabulated in Table C.

TABLE C

| | | | Feed | | | | | | | | |
|---|---|---|---|---|---|---|---|---|---|---|---|
| | | | flow | Distillate | Rotation | | CDA | CDA | impurities | | |
| | T | Vacuum | rate | flow rate | rate | D/F | Assay | Purity | Trans | BMNA | Yield |
| Run | ° C | mbar | ml/min | ml/min | rpm | % | % | % | % | % | % |

Runs A1-A3 (invention) and A4 (comparative)

| | | | | | |
|---|---|---|---|---|---|
| Crude CDA feed for runs A1-A4 | 71 | 88.19 | 0.63 | 7.43 | — |

Distillates obtained from A1-A4

| | | | | | | | | | | | |
|---|---|---|---|---|---|---|---|---|---|---|---|
| A1 | 150 | 3.8 | 20 | 11 | 175 | 55 | 94 | 96.3 | 0.4 | 1.3 | 84 |
| A2 | 150 | 4.1 | 14 | 9.9 | 175 | 71 | 98 | 95.9 | 0.9 | 1.7 | 88 |
| A3 | 180 | 4.2 | 35.3 | 23.6 | 175 | 67 | 95 | 95.1 | 0.6 | 2.8 | 89 |
| A4 | 145 | 5.6 | 7 | 4 | 175 | 57 | 95 | 96.8 | 0.8 | 1.1 | 42 |

Runs B1-B3 (invention) and B4 (comparative)

| Crude CDA feed for runs B1-B4 | | | | | | | 68.8 | 88.7 | 0.6 | 8.0 | — |
|---|---|---|---|---|---|---|---|---|---|---|---|
| B1 | 150 | 3.9 | 15 | 11.1 | 175 | 74 | 94.1 | 95.8 | 0.8 | 1.7 | 88 |
| B2 | 150 | 3.5 | 15.2 | 12.7 | 250 | 83 | 91.9 | 95.6 | 0.9 | 2.0 | 91 |
| B3 | 150 | 3.8 | 16.8 | 12.4 | 250 | 73 | 92.1 | 95.5 | 0.8 | 1.9 | 90 |
| B4 | 150 | 5.7 | 14.1 | 8.2 | 175 | 58 | 93.4 | 97.3 | 0.5 | 1.2 | 47 |

The results tabulated in Table C show that a product of high CDA assay (≥90%) with a good production yield in the range of 80-90% can be achieved by wiped film evaporation of the crude product at a temperature above 150° C., e.g., from 150° C. to 180° C. While good assay results were observed in several runs when a deep vacuum was not created (A4, B4), these runs were shown to result in low productivity/low yield. Therefore, it is beneficial to hold an evaporation run at a low pressure (<4.5 mbar). To get a desirable ratio D/F near 71%, feed flow is maintained in the range of 14-15 ml/min at 150° C. and 35 ml/min at 180° C.

### Example 11

### Purification of Crude Cis-2-Decenoic Acid by Wiped-Film Evaporation (Pilot Scale)

The WFE system for crude purification included a 0.4 m² WFE from “Canzler” and 100 L bottom tank, both made of stainless steel 316. The WFE was heated with thermal oil from an electrical heater (“Lauda”, 48 kW); the bottom tank was not heated. The CDA crude was fed in by a peristaltic pump from a 200 L drum and placed on balances WI-1 to the top of the WFE system. The residue was collected in the bottom tank and was drained at the end of each run. The vapors were condensed in a 0.75 m² stainless steel condenser cooled by water from a cooling tower. The distillate was collected in a 100 L glass distillate receiver and was withdrawn with a diaphragm pump P-3 at the end of each run. The condenser and the distillate receiver were connected to a deep-vacuum dry pump, CXS250 from “Edwards”.

Several exploratory runs were made to define best evaporation conditions for the crude CDA (63.6 kg, assay 67% w/w, HPLC).

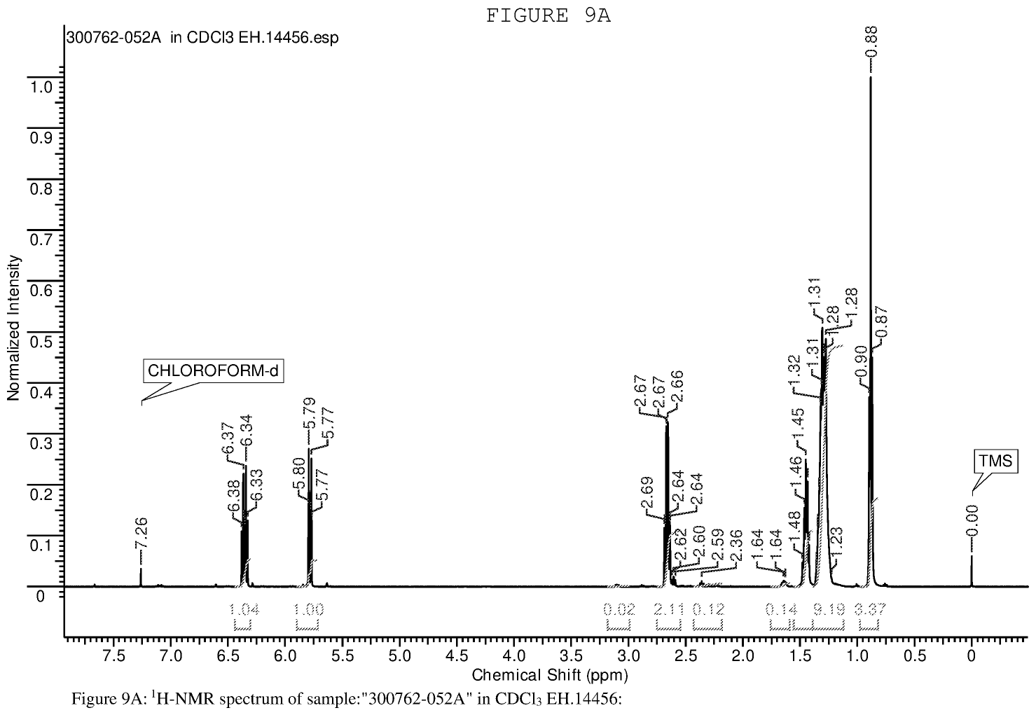

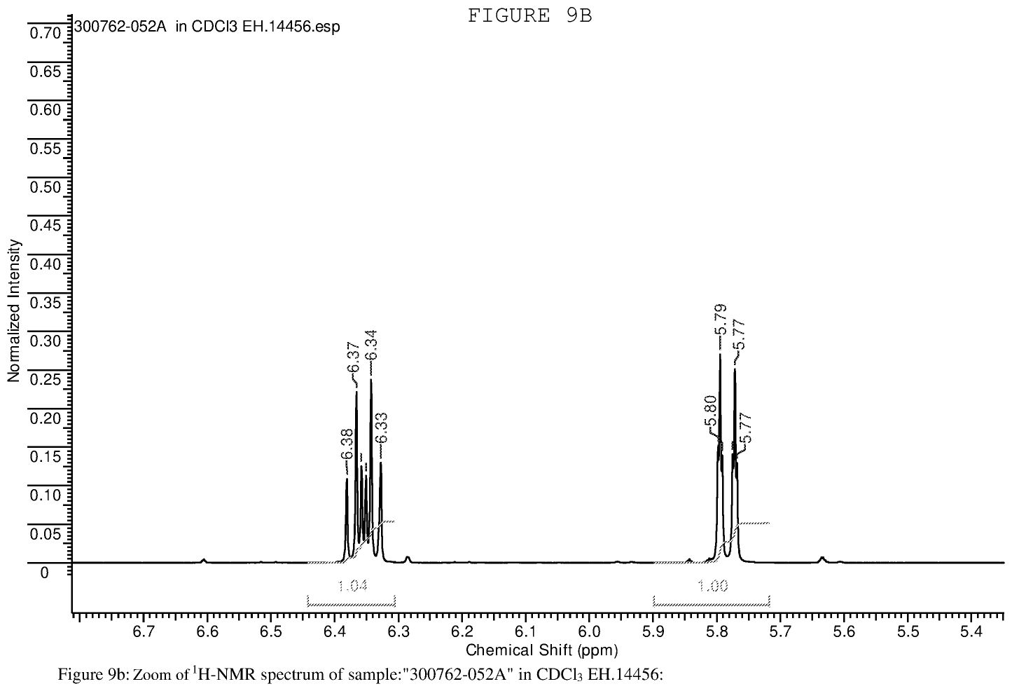

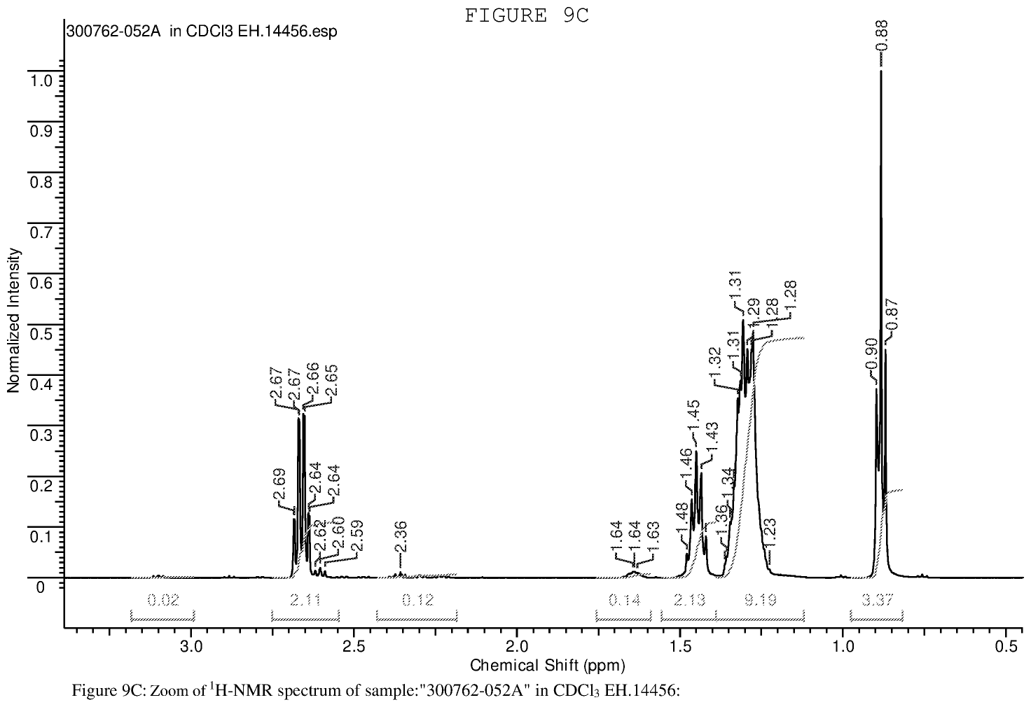

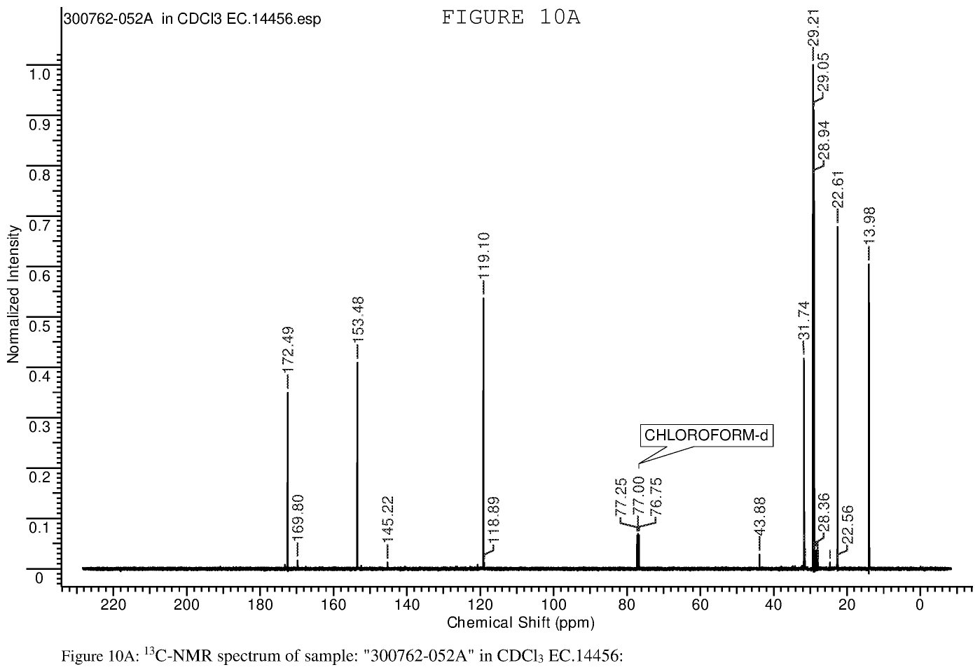

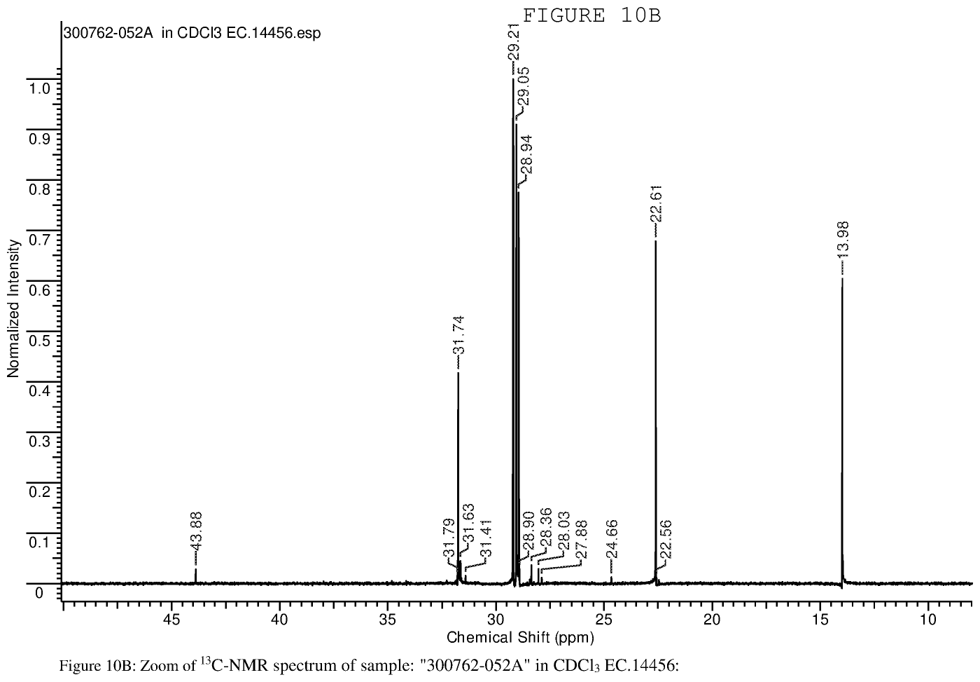

Good results (recovery of pure CDA with Assay ≥90% (by HPLC)) were obtained by carrying out a two pass distillation mode at 175-180° C., 1 mbar, feed rate 27 kg/hr for the 1^{st} pass, (189° C., ˜1 mbar pressure at top) yielding 40 kg CDA (77% assay w/w) and 55 kg/hr for the 2^{nd} pass, (180° C., 1 mbar pressure at top) to yield 23.8 kg in-spec. CDA (98% assay). The bottoms of the 2^{nd} pass (11 kg) were also distilled at 175-180° C. and a feeding rate of 60 kg/hr, yielding 7 kg in-spec CDA (93%). The total yield of the WFE purification was 72% in-spec (>95% assay) CDA from the crude CDA product. FIGS. 9A, 9B and 9C are ¹H-NMR spectra of CDA obtained, and FIGS. 10A and 10B are the 1³C-NMR spectra of CDA obtained.

### Example 12

### Thermal Isomerization of CDA

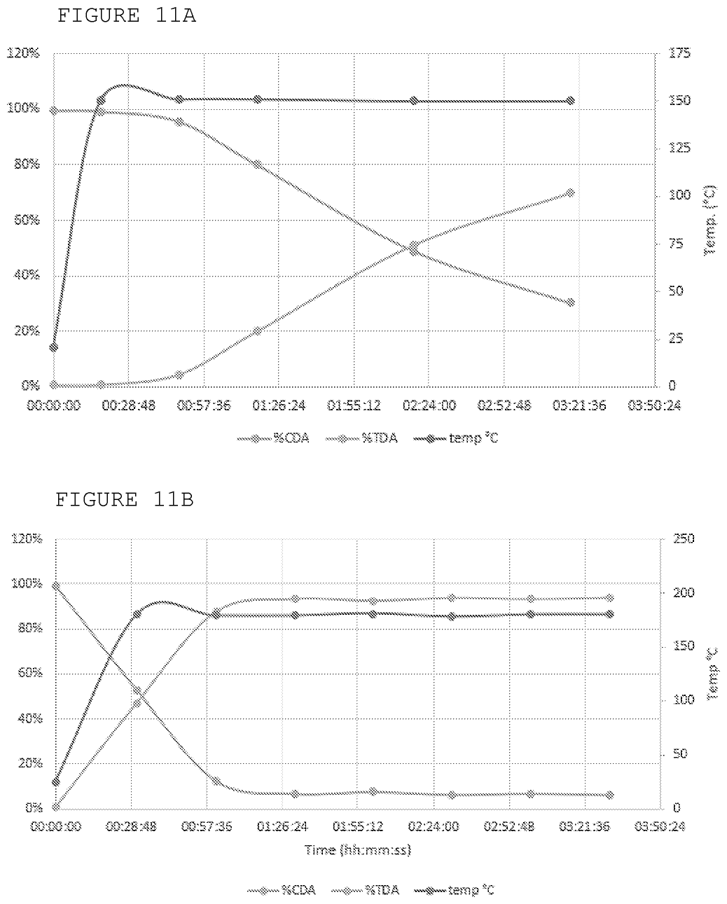

A study was conducted to determine the thermal isomerization of CDA. The CDA was charged into a flask, equipped with a magnetic stirrer, a thermocouple, and a condenser. FIGS. 11A and 11B show isomerization of the cis isomer into the trans isomer (TDA), at 150° C. and 180° C., respectively, at normal pressure, based on sampling at different times and analyzing in HPLC. It is seen that CDA is thermally labile, with very significant isomerization occurring quite rapidly.

1. A method of purification of crude cis-2-alkenoic acid by thermal separation, comprising wiped-film evaporation or vacuum distillation of the crude cis-2-alkenoic acid, under a temperature-reduced pressure profile whereby isomerization of cis-2-alkenoic acid into trans-2-alkenoic acid is minimized.

2. The method according to claim 1, comprising wiped-film evaporation of crude cis-2-alkenoic acid of the formula R—CH═CH—COOH wherein R is a straight alkyl chain CH₃—(CH₂)ₙ—, with 3≤n≤10.

3. The method according to claim 2, wherein the cis-2-alkenoic acid is cis-2-decenoic acid and wherein the wiped-film evaporation is carried out at a temperature of not less than 150° C. and reduced pressure of <5 mbar.

4. The method according to claim 3, wherein the temperature is in the range from 150 to 180° C. and the reduced pressure is 1-5 mbar, whereby isomerization of the cis isomer to the trans isomer is suppressed such that the level of the trans isomer is less than 1.0% by HPLC area.

5. The method according to claim 1, wherein the wiped-film evaporation comprises a step of collecting a condensate and returning it to the feed stream of the wiped-film evaporator.

6. The method according to claim 1, wherein the crude cis-2-alkenoic acid is prepared by a process comprising the steps of:
brominating 2-alkanone to give 1,3-dibromo-2-alkanone; and rearranging the 1,3-dibromo-2-alkanone to the cis-2-alkenoic acid.

7. The method according to claim 1, wherein the crude cis-2-alkenoic acid is prepared by a process comprising the steps of:
rearranging 1,3-dibromo-2-alkanone in an alkaline environment in the presence of a catalytically effective amount of an alkali metal salt of cis-2-alkenoic acid and isolating from the reaction mixture cis-2-alkenoic acid, either in the form of the free acid or in the form of the alkali metal salt.

8. The method according to claim 7, wherein the crude cis-2-alkenoic acid is prepared by a process comprising the steps of:
gradually adding the 1,3-dibromo-2-alkanone to a reaction vessel which was previously charged with an alkaline aqueous solution of Na₂CO₃, K₂CO₃, or a mixture thereof and a catalytically effective amount of an alkali metal salt of cis-2-alkenoic acid, at elevated temperature.

9. The method according to claim 7, wherein the process of preparing the crude cis-2-alkenoic acid comprises separating the reaction mixture into aqueous and organic phases, and working-up the aqueous phase, to recover therefrom cis-2-alkenoic acid, either in the form of the free acid or in the form of the alkali metal salt.

10. The method according to claim 9, wherein the process of preparing the crude cis-2-alkenoic acid comprises working-up the aqueous phase by washing with an organic solvent, followed by phase separation, to obtain a purified aqueous phase.

11. The method according to claim 7, wherein the process of preparing the crude cis-2-alkenoic acid comprises optional dilution of the reaction mixture with water and washing with an organic solvent, followed by phase separation, to obtain a purified aqueous phase.

12. The method according to claim 10, wherein the process of preparing the crude cis-2-alkenoic acid further comprises acidifying the purified aqueous phase to obtain a biphasic medium, comprised of a heavy, salt-containing aqueous phase, and a light organic phase consisting essentially of the cis-2-alkenoic acid in the form of the free acid.

13. The method according to claim 6, wherein the process of preparing the crude cis-2-alkenoic acid comprises rearranging 1,3-dibromo-2-alkanone selected from the group consisting of:

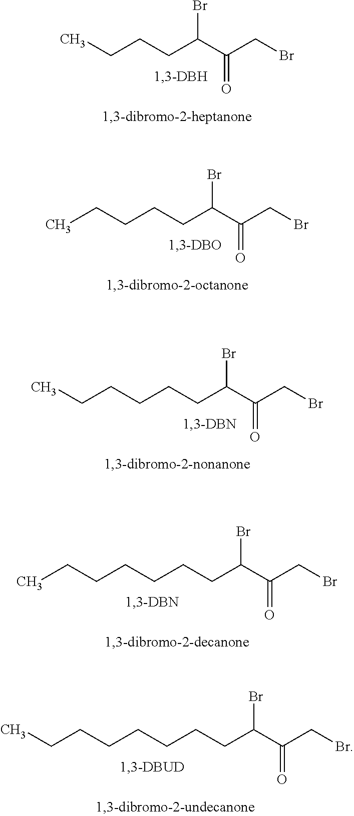

14. The method according to claim 13, wherein the 1,3-dibromo-2-alkanone, used in the rearrangement reaction, is a crude 1,3-dibromo-2-alkanone obtained by the steps of:
brominating the corresponding 2-alkanone selected from the group consisting of 2-heptanone, 2-octanone, 2-nonanone, 2-decanone and 2-undecanone in concentrated hydrobromic acid by the addition of elemental bromine, whereby 1,3-dibromo-2-alkanone is formed in the reaction mixture alongside 3,3-dibromo-2-alkanone;
maintaining the reaction mixture over a hold time adjusted to maximize the interconversion of 3,3-dibromo-2-alkanone to 1,3-dibromo-2-alkanone;
and collecting the crude 1,3-dibromo-2-alkanone by phase separation.

15. The method according to claim 6, wherein the process of preparing the crude cis-2-alkenoic acid comprises rearranging 1,3-dibromo-2-alkanone in the presence of catalytically effective amount of the alkali metal salt of cis-2-alkenoic acid of up to 10 mol % based on 1,3-dibromo-2-alkanone.

16. The method according to claim 8, wherein the process of preparing the crude cis-2-alkenoic acid comprises removing a minor portion of the aqueous phase before or after the aqueous phase is worked-up, and using said portion of the aqueous phase to supply the catalytically effective amount of alkali metal salt of cis-2-alkenoic acid in a rearrangement reaction of the corresponding 1,3-dibromo-2-alkanone.

17. The method according to claim 7, wherein the process of preparing the crude cis-2-alkenoic acid comprises supplying the catalytically effective amount of the alkali metal salt of cis-2-alkenoic acid to the rearrangement reaction in the form of aqueous solution recovered from an earlier rearrangement reaction.

18. The method according to claim 1, wherein the cis-2-alkenoic acid is cis-2-decenoic acid.
